(19) United States
(12) Patent Application Publication
(10) Pub. No.: US 2025/0255585 A1
(43) Pub. Date: Aug. 14, 2025

(54) DEVICE FOR COLLECTING EXHALED AEROSOLS DURING MECHANICAL VENTILATION

(71) Applicant: University of Pittsburgh - Of the Commonwealth System of Higher Education, Pittsburgh, PA (US)

(72) Inventors: Timothy E. Corcoran, Ben Avon Heights, PA (US); Rutuja A. Kulkarni, Pittsburgh, PA (US); Steven G. Kefalos, II, Pittsburgh, PA (US)

(73) Assignee: University of Pittsburgh - Of the Commonwealth System of Higher Education, Pittsburgh, PA (US)

(21) Appl. No.: 19/097,601

(22) Filed: Apr. 1, 2025

Related U.S. Application Data
(63) Continuation of application No. PCT/US2023/075908, filed on Oct. 4, 2023.
(60) Provisional application No. 63/413,445, filed on Oct. 5, 2022.

(51) Int. Cl. A61B 10/00; B01D 45/02; B01D 45/08

(52) U.S. Cl. CPC A61B 10/00; B01D 45/02; B01D 45/08; A61B 2010/0087

(57) ABSTRACT

An impactor sample collection apparatus includes an inlet and a first impaction stage downstream of the inlet, the first impaction stage defining a longitudinal first axis. A second impaction stage is downstream of the first impaction stage, the second impaction stage being offset from the first impaction stage along a second axis that is perpendicular to the longitudinal first axis. An outlet is downstream of the second impaction stage.

### CROSS REFERENCE TO RELATED APPLICATIONS

The present application is a continuation of International Application No. PCT/US2023/075908, filed Oct. 4, 2023, which claims the benefit of U.S. Provisional Application No. 63/413,445, filed Oct. 5, 2022. Each of the applications above is incorporated herein by reference in its entirety.

### ACKNOWLEDGEMENT OF GOVERNMENT SUPPORT

This invention was made with government support under Grant No. 1R21HL163417 awarded by the National Institutes of Health. The government has certain rights in the invention.

### FIELD

The present disclosure pertains to impactor type sample collection devices for collecting exhaled aerosols from mechanically ventilated patients.

### BACKGROUND

Patients physically unable to breathe may need mechanical ventilation using a ventilator to deliver breaths. Mechanically ventilated patients have a relatively high risk of developing pneumonia, known as ventilator-associated pneumonia (VAP). However, detecting the onset of VAP and determining the responsible pathogen often requires invasive procedures such as lung lavage to collect samples of lung secretions. Accordingly, there exists a need for improved systems and methods for collecting samples of airway and deep lung secretions from mechanically ventilated patients.

### SUMMARY

Certain examples of the disclosure pertain to impactor type sample collection devices for collecting exhaled aerosols from mechanically ventilated patients. In a representative example, an impactor sample collection apparatus comprises an inlet; a first impaction stage downstream of the inlet, the first impaction stage defining a longitudinal first axis; a second impaction stage downstream of the first impaction stage, the second impaction stage being offset from the first impaction stage along a second axis that is perpendicular to the longitudinal first axis; and an outlet downstream of the second impaction stage.

In another representative example, an impactor sample collection apparatus comprises an inlet; a first impaction stage downstream of the inlet, the first impaction stage configured to receive a removable sample collection container; a second impaction stage downstream of the first impaction stage, the second impaction stage configured to receive a removable sample collection container; and an outlet downstream of the second impaction stage.

In another representative example, a method comprises collecting aerosolized liquid droplets from exhaled breaths of a patient in a first impaction stage of an impactor sample collection apparatus, the aerosolized liquid droplets collected in the first impaction stage having a first diameter range; and collecting aerosolized liquid droplets from exhaled breaths of the patient in a second impaction stage of the impactor sample collection apparatus, the aerosolized liquid droplets collected in the second impaction stage having a second diameter range that is less than the first diameter range.

In another representative example, an impactor sample collection apparatus comprises a first conduit portion comprising an inlet, the first conduit portion extending along a first axis; the first conduit portion comprising a first impaction stage downstream of the inlet and aligned along the first axis; a second conduit portion in fluid communication with the first conduit portion and extending along a second axis, the second axis forming an angle of 10° to 90° with the first axis of the first conduit portion; and a second impaction stage downstream of the first impaction stage and spaced apart from the first impaction stage along the second axis.

The foregoing and other objects, features, and advantages of the disclosed technology will become apparent from the following detailed description, which proceeds with reference to the accompanying figures.

### BRIEF DESCRIPTION OF THE DRAWINGS

FIG. 1 schematically illustrates a sample collection apparatus connected to a mechanical ventilator between the ventilator and a ventilated patient, as well as diagnostic tools including a DNA/RNA sequencing device and an assay panel.

FIG. 2 schematically illustrates a representative example of a multistage impactor sample collection apparatus.

FIGS. 3A and 3B illustrate example configurations of a second impaction stage of a sample collection apparatus.

FIG. 4 is a perspective view of a prototype sample collection apparatus according to FIG. 2.

FIG. 5 is a perspective view illustrating the sample collection container detached from the sample collection apparatus of FIG. 4.

FIG. 6 is a perspective view of another example of a sample collection apparatus.

FIG. 7 illustrates the sample collection apparatus of FIG. 6 in a disassembled state.

FIG. 8 is a perspective view illustrating a funnel and a sample collection container of the sample collection apparatus of FIG. 6.

FIG. 9 is a perspective view of an example nozzle of the first impaction stage of the sample collection apparatus of FIG. 6.

FIG. 10 is a perspective view of an example nozzle of the second impaction stage of the sample collection apparatus of FIG. 6.

FIG. 11 is a perspective view of the second impaction stage of the sample collection apparatus of FIG. 6 as viewed from the outlet.

FIG. 12 is a perspective view of the nozzle of the second impaction stage including an impaction member.

FIGS. 13A-13C illustrate traces of volume, proximal airway pressure, and inhalation and exhalation flow rate, respectively, for a baseline ventilator-test lung circuit and a circuit including a sample collection apparatus (collector) in the exhalation line.

FIG. 14A is a schematic diagram of perfused and ventilated lungs.

FIGS. 14B and 14C are photographs of ex vivo human lungs for testing of the sample collection apparatus examples described herein.

FIG. 15 illustrates a test setup with the sample collection apparatus of FIG. 6 coupled between a mechanical ventilator and a pair of ex vivo lungs.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this disclosure and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But these terms are not intended to imply absolute relationships, positions, and/or orientations. For example, with respect to an object, an “upper” surface can become a “lower” surface simply by turning the object over. Nevertheless, it is still the same object.

Unless otherwise indicated, all numbers expressing angles, dimensions, quantities of components, forces, moments, percentages, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited.

Although there are alternatives for various components, dimensions, parameters, operating conditions, etc., set forth herein, that does not mean that those alternatives are necessarily equivalent and/or perform equally well. Nor does it mean that the alternatives are listed in a preferred order unless stated otherwise.

As used herein, values and/or relationships modified by the term “substantially” mean 10% of the stated value and/or relationship. “Substantially perpendicular” means an angle of 800 to 100° relative to a reference. “Substantially parallel” means an angle of ±10° relative to a reference.

### Example 1: Multistage Impactor Sample Collection Apparatus

The sample collection devices described herein are configured to be placed in communication with the exhalation limb of a mechanical ventilator and to collect aerosolized liquid droplets, such as water droplets, entrained in the exhaled breath of the patient. The aerosolized liquid droplets can include, for example, water, mucus, lung surfactant (e.g., proteins and lipids), etc., in various concentrations depending on the location of origin. Such aerosolized liquid droplets are also referred to herein as “aerosols.” The sample collection devices described herein are further configured to collect aerosols having specified sizes in different parts of the apparatus. In certain examples, aerosolized liquid droplets having sizes within specified ranges originate at different locations within the human airway as further described below. The sample collection devices described herein can include one or a plurality of impactor type sample collection stages referred to hereinafter as “impaction stages.” The impaction stages are coupled together in series fluid communication along a tortuous flow path defined by a conduit. In certain examples, the conduit can comprise a unitary body or a plurality of pipes and/or conduit segments coupled together.

The flow path defined by the conduit can include one or more curves, elbows, T-junctions, diameter changes such as diameter reductions (e.g., nozzles), etc., configured to change the direction of flow of gases and aerosolized liquid droplets flowing through the device. The impaction stages can be arranged along the flow path to utilize gravity as well as the momentum of the entrained aerosolized liquid droplets to sort and collect aerosols of specified size and/or mass at specified impaction stages. This advantageously provides the ability to collect samples of a patient's lung secretions originating from different locations in the airway using a single device, and without the need for invasive sampling procedures such bronchoalveolar lavage. Moreover, samples can be collected at relatively short time intervals, allowing near continuous monitoring of at-risk patients and early detection of ventilator-associated-pneumonia (VAP).

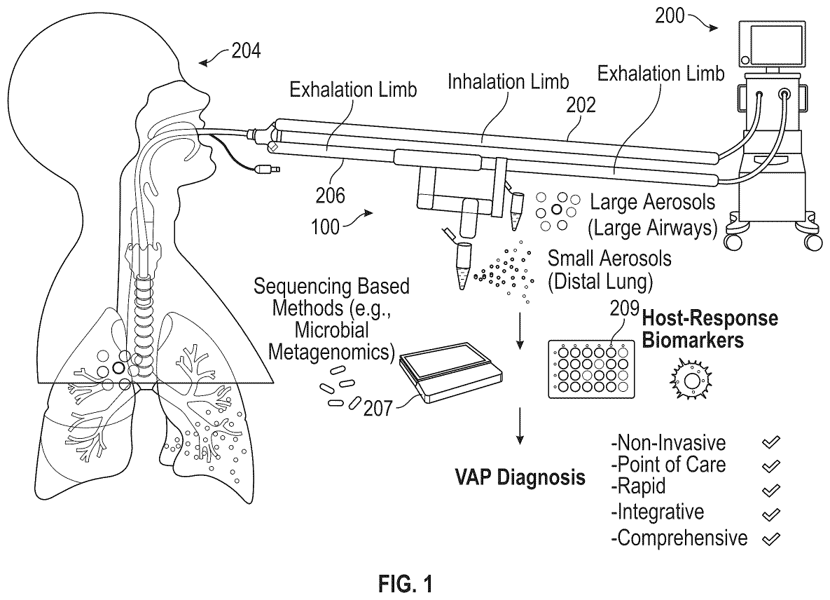

FIG. 1 schematically illustrates a representative example of a sample collection device 100 as described herein coupled to a mechanical ventilator 200. An inhalation limb 202 of the ventilator is shown leading to an intubated patient 204 being ventilated with the ventilator. The sample collection device 100 is coupled in line with the exhalation limb 206 of the ventilator. As such, breaths exhaled by the patient flow through the sample collection device 100, which collects samples of aerosolized secretions from different parts of the patient's lung as described below.

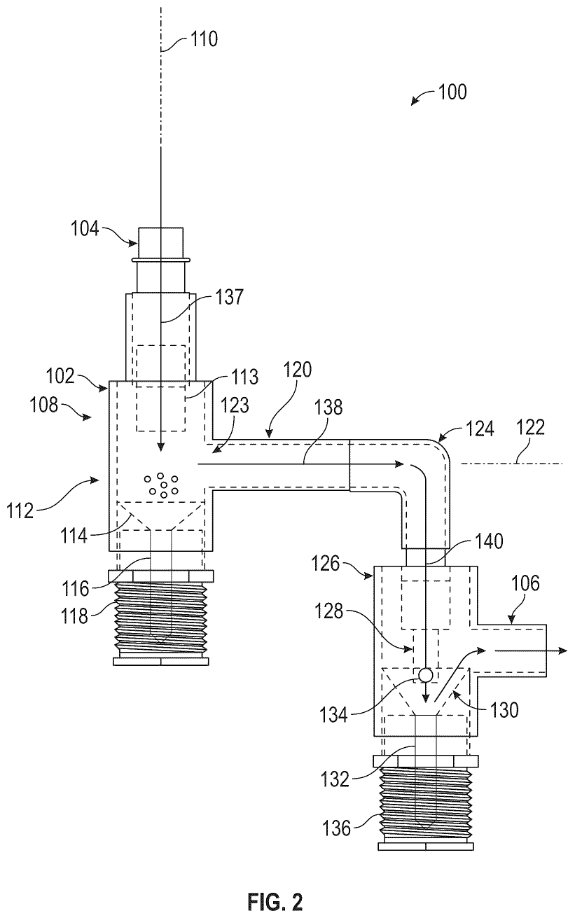

FIG. 2 illustrates a representative example of the impactor sample collection device 100. The device 100 can comprise a conduit 102 defining a flow path between an inlet 104 (e.g., an inlet fitting configured to be coupled to the upstream portion of a ventilator exhalation limb) and an outlet 106 (e.g., an outlet fitting configured to be coupled to the downstream portion of a ventilator exhalation limb). The conduit 102 can comprise a first conduit portion 108, which can include the inlet 104 at an upstream portion thereof. The first conduit portion 108 can extend along a first axis 110, which in use can be oriented in the direction of gravity. The first conduit portion 108 can comprise a first impaction stage generally indicated at 112. The first impaction stage 112 can comprise a nozzle 113 or other reduced diameter structure configured to accelerate gas flowing through the first conduit portion along the first axis 110. In certain examples, the inner diameter of the first conduit portion 108 upstream of the nozzle can be 20 mm to 40 mm, such as 25 mm to 40 mm, 30 mm to 40 mm, etc. In one particular example, the inner diameter of the first conduit portion 108 upstream of the nozzle 113 can be 33 mm. The outlet of the nozzle 113 can have an inner diameter of 10 mm to 30 mm, such as 10 mm to 25 mm, 12 mm to 25 mm, 15 mm to 25 mm, 15 mm to 22 mm, etc. A funnel 114 can be positioned downstream of and axially aligned with the nozzle 113. In certain examples, the nozzle 113 and the funnel 114 can be spaced apart by a distance of 5 mm to 40 mm, such as 5 mm to 30 mm, 10 mm to 30 mm, 5 mm to 25 mm, 10 mm to 25 mm, etc., along the axis 110. In a particular example, the end of the nozzle 113 and the top of the funnel 114 can be spaced apart by 18 mm. The first impaction stage 112 can further comprise a sample collection container 116 with an open end and a closed end, such as an Eppendorf tube, positioned in the flow path beneath the funnel 114. The sample collection container 116 can be held in place by a retainer such as a threaded plug 118.

The conduit 102 can further comprise a second conduit portion 120 in fluid communication with the first conduit portion 108 and extending from the first conduit portion 108 along a second axis 122. In certain examples, the second axis 122 can form an angle of 10° to 90° with the first axis 110. An inlet 123 of the second conduit portion 120 can be above the collection container 116 along the first axis 110. In the illustrated example, the inlet 123 of the second conduit portion 120 is located between the nozzle 113 and the funnel 114 along the first axis 110. In other examples, the outlet of the nozzle 113 can be above the inlet 123 or can extend below the upper edge of the inlet 123 depending, for example, on the spacing between the nozzle 113 and the funnel 114. In certain examples, the second conduit portion 120 can form a T-junction with the first conduit portion 108.

The second conduit portion 120 can comprise a bend 124 (also referred to as a curve or elbow) downstream of the second conduit portion inlet 123. The device can further comprise a second impaction stage 126 downstream of the bend 124. In certain examples, the bend 124 can be a 90° bend such that a longitudinal axis of the second impaction stage 126 is parallel or substantially parallel to the first axis 110. The second impaction stage 126 can also be offset at least partially from the first impaction stage 112 along the first axis 110 (e.g., lower than the first impaction stage along the first axis 110). Thus, the second impaction stage 126 can be offset from the first impaction stage 112 along two mutually orthogonal axes (e.g., the first axis 110 and the second axis 122).

The second impaction stage 126 can comprise a nozzle 128, a funnel 130 downstream of the nozzle 128, and a sample collection container 132 downstream of the funnel 130. In certain examples, the inner diameter of the second conduit portion 120 can be any of the diameter ranges given above with reference to the first conduit portion 108. In certain examples, the outlet diameter of the nozzle 128 can be 1 mm to 20 mm, such as 1 mm to 15 mm, 3 mm to 15 mm, etc. In certain examples, the nozzle 128 can be partially received in the funnel 130. In certain examples, an impaction member 134 can be positioned in the flow path of the second impaction stage, such as at the outlet of the nozzle 128. The sample collection container 132 (e.g., an Eppendorf tube) can comprise an open end and a closed end, and can be positioned in the flow path beneath the funnel 130. The sample collection container 132 can be held in place by a threaded plug 136 or other retainer. In certain examples, the impaction member 134 can be a cylinder extending across the diameter of the nozzle 128, a ball or sphere, or another shape providing additional surface area on which aerosolized liquid droplets can impact, land, collect, and/or condense. In certain examples, the first impaction stage can also include an impaction member positioned in the flow path of the first conduit portion depending on the particular characteristics sought.

The outlet fitting 106 can be in fluid communication with the second impaction stage 126. In certain examples, the outlet fitting 106 can be above the sample collection container 132. The outlet fitting 106 can be configured for coupling to a downstream portion of the exhalation limb of a ventilator such as the ventilator 200.

In use, exhaled breaths from the ventilated patient 204 can flow through the upstream portion of the exhalation limb of the ventilator circuit to the inlet 104 of the sample collection device 100 and can flow through the first impaction stage. More particularly, the exhaled gas can be accelerated along the first axis 110 by the nozzle 113 in the direction of arrow 137 (FIG. 2) and can flow through the funnel 114 into the sample collection container 116. Aerosolized liquid droplets having a diameter of 20 μm to 1,000 μm or greater, such as 20 μm to 500 μm, can be deposited on the walls of the nozzle 113, the funnel 114, and/or the sample collection container 116. In certain examples, the momentum of aerosols in these diameter ranges in the downward direction along the first axis 110 increases the number of particles that are deposited on the surfaces of the first impaction stage and reduces the number of particles in these diameter ranges that remain entrained in the airflow out of the first impaction stage. The deposited droplets can flow downwardly into the sample collection container 116 to form a liquid sample. In certain examples, aerosolized liquid droplets in the diameter ranges above are likely to have originated in the proximal lung, throat, and/or mouth of the patient.

After flowing through the first impaction stage 112 the exhaled gas can flow through the inlet 123 into the second conduit portion 120 in the direction of arrow 138. The bend 124 of the second conduit portion 120 can direct the gas flow downwardly toward the second impaction stage 126 in the direction of arrow 140. The gas can be accelerated by the nozzle 128 through the funnel 130 and into the sample collection container 132. In certain examples, the second impaction stage 126 can be configured to capture aerosolized liquid droplets having a diameter of 1 μm to 19 μm, such as 1 μm to 10 μm. The aerosolized liquid droplets can be deposited on the surfaces of the nozzle 128, the funnel 130, the impaction member 134, and/or the walls of the sample collection container 132. The deposited droplets can flow downwardly into the sample collection container 132 to form a liquid sample. In certain examples, aerosolized liquid droplets having diameters within the ranges given above for the second impaction stage are likely to have originated mostly in the distal lung.

Over time, aerosolized liquid droplets deposited on the surfaces of the first impaction stage 112 can flow and collect at the bottom of the sample collection container 116. Similarly, aerosolized liquid droplets deposited on the surfaces of the second impaction stage 126 can flow and collect at the bottom of the sample collection container 132. The samples can be collected at selected intervals and analyzed to determine the presence or absence of pathogens, such as by using any of a variety of sequencing-based methods such as rapid bacterial, viral, and/or fungal DNA and/or RNA sequencing systems. The samples can also be analyzed to detect the presence or absence of biomarkers indicative of an inflammatory response as further described in the examples below.

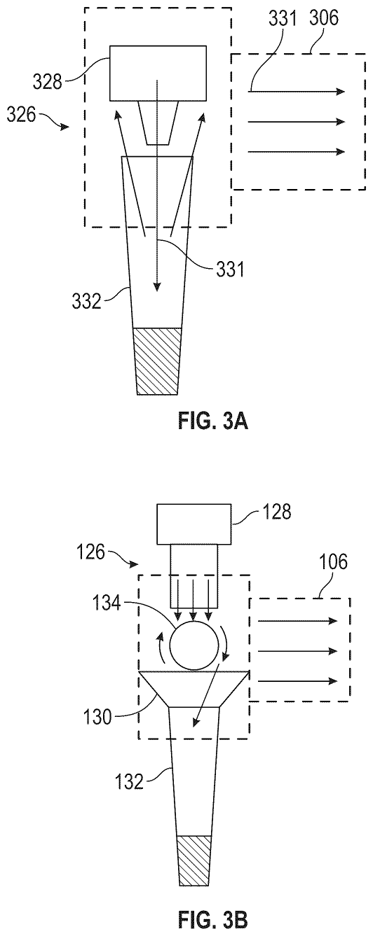

FIG. 3A illustrates airflow through another example of an impaction stage 326 without a funnel. The impaction stage 326 of FIG. 3A can be implemented at either the location of the first impaction stage or the second impaction stage, but can be particularly suited for use as the second impaction stage. The impaction stage 326 can include a nozzle 328 positioned above and axially aligned with a sample collection container 332. Arrows 331 indicate the general flow path of exhaled air through impaction stage 326 into the sample collection container 332 and out through the outlet portion 306. FIG. 3B illustrates an alternative configuration of the second impaction stage 126 in which the impaction member 134 is configured to rotate as air flows around it.

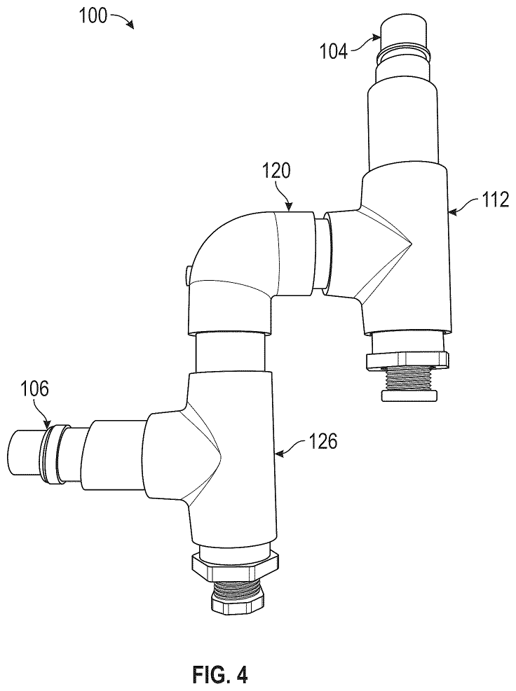

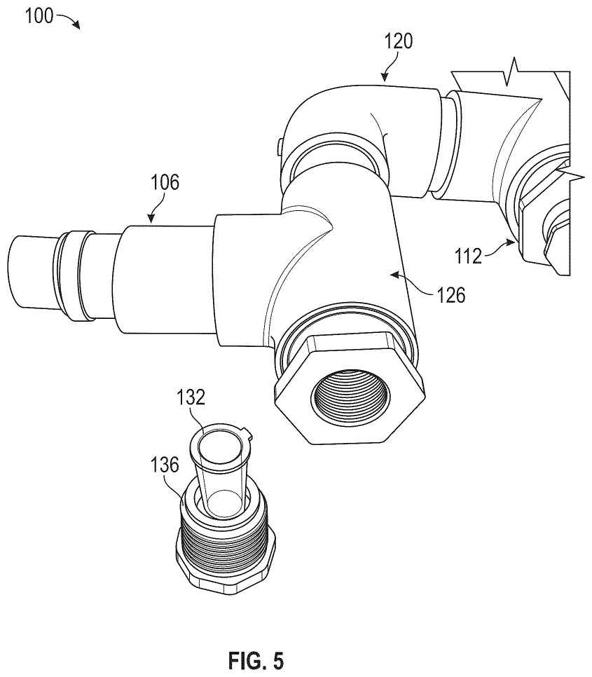

FIG. 4 illustrates a working example of an impactor sample collection device according to the present disclosure in which the inlet is shown at the upper right of the figure and the outlet is at the lower left. FIG. 5 illustrates the sample collection container of the second impaction stage separated from the device.

In other examples, the devices described herein can include more than two impaction stages, such as three impaction stages, four impaction stages, etc., or a single impaction stage. In certain examples, the flow path through the device can be configured differently and may include, for example, more or fewer conduit portions, more or fewer curves, curves of different angles, etc. Additionally, the spacing between various components such as between the nozzles, the funnels, and/or the sample collection containers of the impaction stages can vary according to the particular characteristics sought. In certain examples, an electrical charge can be applied to one or more selected components of the impaction stages, such as to the nozzles or the impaction surfaces such as impaction member 134, to enhance collection efficiency. For example, the selected component(s) can be made from an electrically conductive material (e.g., a metal such as aluminum, copper, steel, etc.) and connected to one terminal (e.g., the positive or negative terminal) of a battery. In one particular example, an electrical charge can be applied to the nozzle 128 and/or the funnel 130 of the second impaction stage to enhance collection efficiency of the relatively small diameter aerosolized liquid droplets of the distal lung.

Any or all of the impactor sample collection device examples described herein can provide a number of significant advantages over known devices and methods of sampling respiratory excretions from mechanically ventilated patients. For example, the structure of the device allows the collection of aerosolized liquid droplets directly from a ventilated patient's exhaled breaths in a non-invasive manner. The devices described herein can also be made compatible with most if not all commercially available mechanical ventilators. The flow path length, the diameters of the various portions of the conduit, the nozzle diameters, etc., can also be specified to maintain pressure and volume flow rates in the exhalation limb of a ventilator within specified limits. Stated differently, the devices described herein can be configured so as not to interfere with the normal operation of the ventilator.

Moreover, the arrangement of the first and second impaction stages facilitates the separate collection of aerosolized excretions originating from different parts of a patient's respiratory tract. Thus, the first impaction stage of the devices described herein can collect aerosolized droplets from a patient's breath originating primarily in the large bronchial airways and having a first, relatively large diameter range, while the second impaction stage can collect aerosolized droplets originating primarily in the lower respiratory tract and having a second, relatively small diameter range. This facilitates the simultaneous and continuous monitoring of both the upper respiratory tract and the lower respiratory tract for signs of infection (e.g., VAP), inflammation, and/or other conditions. The devices described herein can also be surprisingly efficient. Working examples of the devices described herein have collected usable sample volumes in both the first and second impaction stages within one hour.

Further details of the systems and methods described herein are given in the additional examples below.

### Example 2: Sample Collection Apparatus with Nozzles and Seals

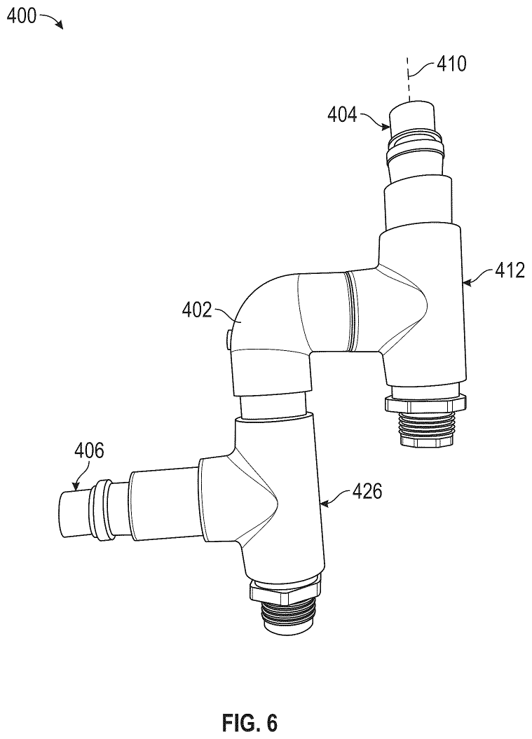

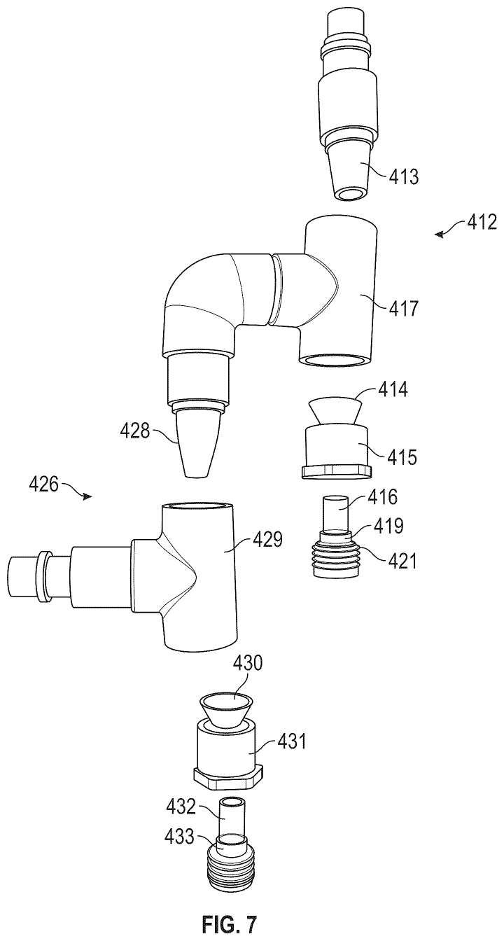

FIG. 6 illustrates another example of a multistage impactor sample collection device 400. The sample collection device 400 is similar to the sample collection device 100, and similar reference numbers in FIG. 6 indicate similar features in the previous examples. The sample collection device 400 includes a conduit 402 defining a flow path between an inlet 404 and an outlet 406 and comprising a first impaction stage 412 and a second impaction stage 426 (also referred to as first and second “sample collection stages”). Referring to FIG. 7, as in the other examples described herein the first impaction stage 412 can comprise a nozzle 413 configured to accelerate gas flowing through the conduit 402 along the first axis 410 (FIG. 6). The outlet of the nozzle 413 can have any of the diameter ranges given herein, such as 10 mm to 20 mm or 10 mm to 15 mm. A funnel 414 can be positioned downstream of and axially aligned with the nozzle 413. In certain examples, the funnel 414 can be received in or coupled to a funnel holder 415 (also referred to as a funnel base member), which can be insertable into a pipe or conduit segment 417 (e.g., a T-junction) that forms part of the first impaction stage 412. The nozzle 413 can be received in a top opening of the conduit segment 417.

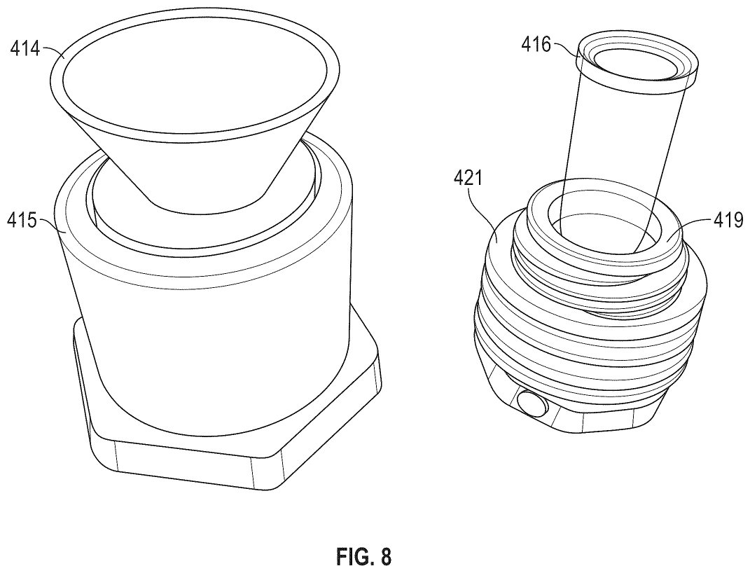

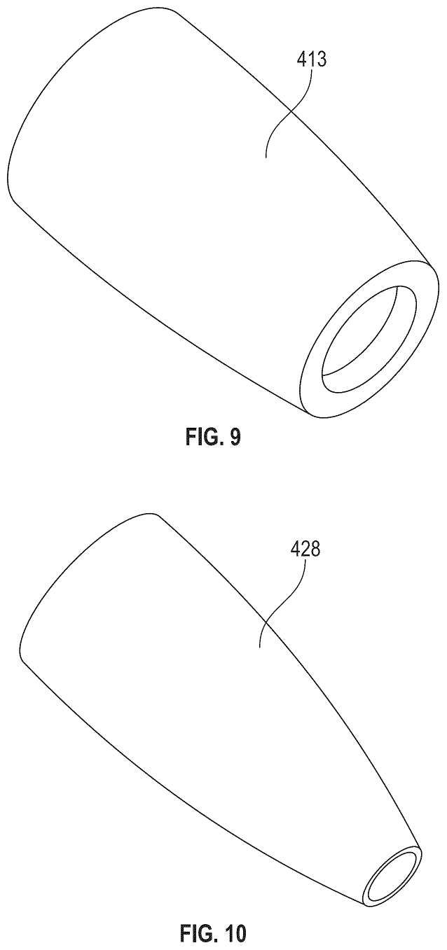

A sample collection container 416 (e.g., an Eppendorf tube) can be received in or coupled to a container holder 419 (also referred to as a threaded plug and a container base member). In some examples, the container holder 419 can comprise threads and can be received in a lower opening of the conduit segment 417 by threading the container holder 419 into the conduit segment 417. The container holder 419 can comprise one or a plurality of sealing members configured as O-rings 421 positioned around the circumference of the container holder 419. The O-rings can seal the connection between the container holder 419 and the funnel holder 415 (and thus with the conduit segment 417) to reduce gas leakage out of the first impaction stage 412 during use. Representative examples of the funnel 414 coupled to the funnel holder 415 and the sample collection container 416 coupled to the container holder 419 are illustrated in FIG. 8. A representative example of the first stage nozzle 413 is shown in FIG. 9.

Returning to FIG. 7, the second impaction stage 426 can comprise a nozzle 428 oriented downwardly toward a funnel 430. The nozzle 428 can be received in the top opening of a conduit segment 429 (e.g., a T-junction) and the funnel 430 can be received in the bottom opening of the conduit segment 429. The funnel 430 can be coupled to a funnel holder 431 similar to the funnel 414. In certain examples, the funnel 430 and the funnel holder 431 can be similar to and/or interchangeable with the funnel 414 and the funnel holder 415. A sample collection container 432 can be received in or coupled to a container holder 433, which can be configured to be threaded or press fit into the funnel holder 431. The container holder 433 can also comprise one or a plurality of sealing members such as O-rings 421 to seal the connection between the container holder 431 and the funnel holder 431 (and thus with the conduit segment 429). In certain examples, the container 432 and the container holder 433 can be similar to and/or interchangeable with the container 416 and the container holder 419 of the first impaction stage. The sample collection containers can be removable by unthreading the bases from the respective impaction stages, and in some examples the Eppendof tubes can be removable form the bases.

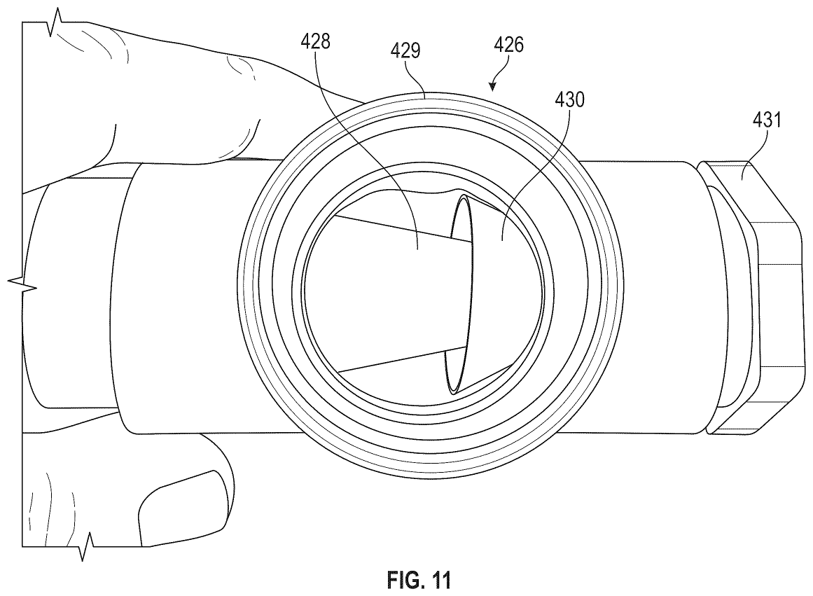

A representative example of the second stage nozzle 428 is shown in FIG. 10. The outlet diameter of the second stage nozzle 428 can be smaller than the outlet diameter of the first stage nozzle 413. The outlet of the nozzle 428 can have any of the diameter ranges recited herein, such as 5 mm to 15 mm or 5 mm to 10 mm.

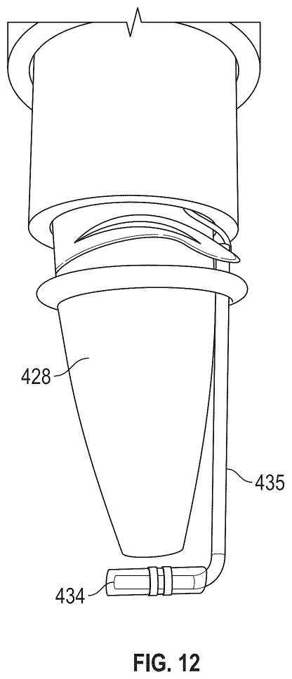

With reference to FIG. 11, as in the previous examples the nozzle 428 can extend into the funnel 430.

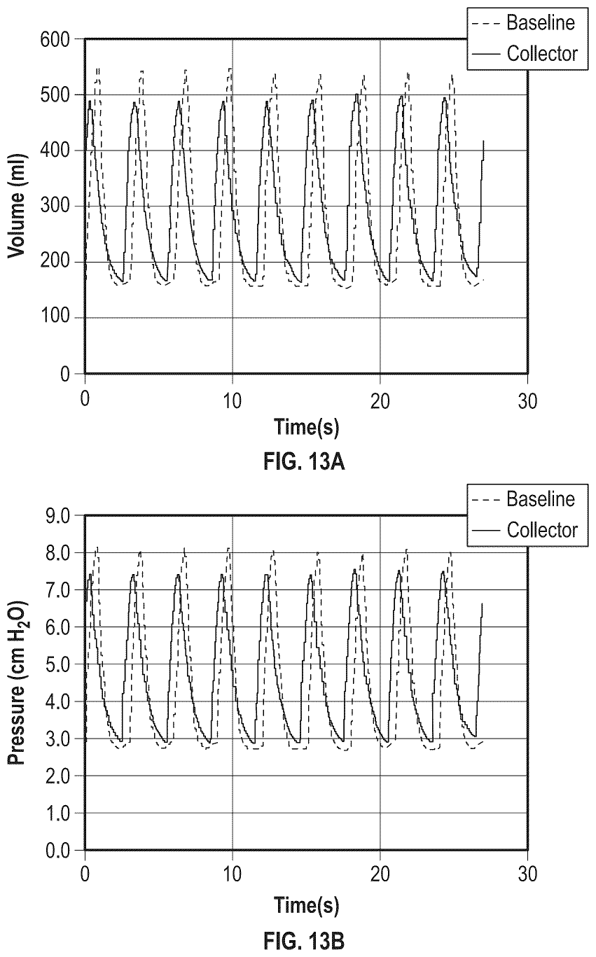

The second impaction stage 426 can also comprise an impaction member positioned in the conduit across which exhaled gas can flow. The impaction member can be positioned in the nozzle 428 similar to the example shown in FIG. 2, downstream of the nozzle 428, upstream of the funnel 430, and/or in the funnel 430. For example, FIG. 12 illustrates an example in which an impaction member 434 is positioned downstream of the nozzle 428 adjacent the outlet of the nozzle 428. In the assembled state the impaction member 434 can be positioned in the funnel 430. In FIG. 12 the impaction member 434 is shown coupled to an arm 435 that is secured to the conduit above the nozzle 428, but in other examples the impaction member can be coupled to the walls of the conduit and/or the funnel 430 at the selected location relative to the nozzle 428. For example, the impaction member 434 can be integrally formed with the funnel 430. The impaction member 434 can also be positioned downstream of the funnel 430. In some examples the impaction member 434 can comprise an electrically conductive material (e.g., a metal such as aluminum, copper, steel, etc.). The impaction member 434 can be configured such that an electric charge can be applied to the impaction member to attract and increase deposition of aerosols in the exhaled air stream on the surface of the impaction member. Additionally, any of the other components and/or interior surfaces of the device such as the funnels, nozzles, etc., can also comprise conductive materials and can be electrically charged.

In yet other examples, the impaction member can include an array of pins or other members disposed in the flow path of the second impaction device, and/or a mesh positioned in the flow path. The first impaction stage can also include any of the impaction members described herein.

In some examples, the sample collection container and the associated base member can be a one-piece unitary construction. In yet other examples, the sample collection container can include one or a plurality of sealing members and need not include a base.

Certain examples of the sample collection devices described herein can include surface coatings on interior surfaces of the device, such as on the nozzles, funnels, the impaction member, etc. Examples of coatings can include lubricious coatings, hydrophobic material coatings such as hydrophobic silicone polymers and the like. Example coatings can include polyvinylpyrrolidone (PVP), polydimethylsiloxane (PDMS), polytetrafluoroethylene (PTFE), etc.

The sample collection devices described herein can be incorporated into a variety of point of care systems including one or more of a sample collection apparatus, a DNA and/or RNA sequencing device such as the device 207 of FIG. 1, and/or a laboratory assay panel (e.g., panel 209 of FIG. 1) for detecting and/or quantifying biomarkers of respiratory pathogens, host defense biomarkers, and/or indicators of lung injury or inflammation that can be read using a small plate reader. In some examples the assay panel can be a polymerase chain reaction (PCR) assay panel. A representative point of care system can include a sample collection apparatus such as any of the collection apparatus described herein, a sequencing device for real-time identification of pathogens based on their genetic material (e.g., metagenomic screening devices such as the MINION® device from Oxford Nanopore Technologies, UK), and a custom panel for quantifying biomarkers of host defense and/or lung injury that can be read using a plate reader. The sample collection apparatus can be placed in the exhalation limb of the ventilator circuit (e.g., ventilator 200 in FIG. 1) and can collect samples of aerosols from the lungs of a ventilated patient. The samples from the first and/or second impaction stages of the sample collector can be collected, and genetic material present in the samples can be sequenced with the sequencing device to determine the presence of pathogens. Biomarkers indicative of host defense, immunity, and/or lung injury can also be analyzed. Treatment(s) can then be administered to the ventilated patient based at least in part on the sequencing and/or analysis results, such as antibiotics in the case of bacterial infection.

Any of the sample collection devices described herein can be included in various kits comprising, for example, a sample collection device, a DNA and/or RNA sequencing device, an assay panel, and/or various consumable items or replacement items such as additional sample collection containers. Sample collection devices as described herein can also be included with mechanical ventilators, and can be permanently connected to the exhalation limbs or attachable and detachable from the exhalation limbs.

### Example 3: Sample Collection Apparatus Prototype Performance

Bench testing includes measurements of aerosol collection efficiency and measurements of air leak during mechanical ventilation using the impactor sample collection apparatus examples described herein. Efficiency measures are made in a closed circuit with an upstream nebulizer generating a saline aerosol with a median diameter approximately 4 μm conveyed through the aerosol collector by a continuous 20 L/min air flow. The size of the aerosol matches the size range we anticipate is being emitted from the deep lung. The second stage is designed to capture this aerosol size range. The current collector has a collection efficiency of 8.5±2.7% (n=8). Bench testing with a mechanical ventilator and a mechanical test lung is performed to assess air leak from device when operating with peak end expiratory pressures (PEEP) of 5-15 cm of H₂O. Sealing the pipe components that house the aerosol collection stages can reduce air leaks.

The inventors performed a test using an ex vivo lung model. This involves the use of human lungs, removed for transplant, but found to be unsuitable when arriving at the treatment center. These lungs are entered into research protocols. The goal of this testing is to compare device-collected aerosol samples to lavage fluid samples collected directly from the lung. In these studies, the aerosol collector was placed into the exhalation line of a mechanical ventilator and the lungs were ventilated over a 3-hour period. Importantly the device did not interfere with ventilator operation.

Human subject testing has enrolled 6 healthy participants who have provided 16 exhaled aerosol samples by exhaling through the collector for one hour. These samples have been utilized for proof-of-concept experiments. The inventors measured protein and surfactant protein-D (SPD) content in 9 pairs (first and second stage) of samples collected from study participants. Volume recovered from the first (large aerosol) stage ranged 7.5-17.5 μL, protein was detected in 8/9 samples. No SPD was detected. This is the anticipated result since the first collector stage is intended to collect large aerosols which originate in the upper airways which have significant amounts of protein but limited or no SPD. Volume recovered in the second stage ranged 0-16.5 μL. 5/9 samples (all of which had volumes of 8.5 μL or greater) had detectable protein content and 2/9 samples had measurable concentrations of SPD, indicating deep lung origin. Outcomes here are as expected.

Preliminary metagenomic studies of batched samples from two participants were also performed. Metagenomic analysis measures DNA within a sample to identify bacterial, viral, or fungal community members. 1/2 samples indicated normal flora that would be anticipated in a sample originating from the lung. The ultimate application of this device will be the detection of pathogens in mechanically ventilated patients.

Some examples of the device can include three-dimensionally (3D) printed nozzles, intended to improve the performance of the device without significantly increasing flow resistance. Some examples of the device can include surface coatings on interior surfaces of the device to improve collection efficiency. In some examples, an electrostatic charge can be applied to certain surfaces and/or regions of the device to improve collection efficiency. Goal efficiency is 20%.

### Example 4: Research and Applications of Impactor Sample Collection Apparatus

### Introduction

The inventors have developed a non-invasive, point of care system for collecting and analyzing exhaled aerosols from the lungs of mechanically ventilated patients that can provide rapid diagnosis of ventilator-associated pneumonia (VAP) through comprehensive, metagenomic screening for bacterial, viral, or fungal pathogens integrated with host-response biomarkers of innate immunity and lung injury. This system can facilitate safe, frequent, non-invasive, convenient sampling of the lower respiratory tract. It can facilitate effective surveillance for VAP, allowing for rapid, point of care diagnosis without the need for extensive microbiologic laboratory workups, and help guide timely treatment decisions against causal pathogens to prevent lung injury and death. It can also provide a valuable research tool for studying the kinetics of lung injury, repair, and resolution.

Integration of microbial metagenomics with host-response biomarkers can allow for differentiation of active infection from quiescent colonization, thus helping avoid unnecessary antibiotics that contribute to drug resistance and increase risk for secondary infections. This system can also facilitate the rapid identification of new or locally endemic pathogens in facilities with limited laboratory capabilities and would provide a valuable tool for studying lung injury during mechanical ventilation. This disclosure is within the portfolio category of respiratory health, the topic area of respiratory health, and the prevention continuum of care. One goal is to prevent lung injury caused by infection.

The point of care system the inventors envision can utilize three technologies: (1) a small passive aerosol collector that can be placed in the exhalation limb of the ventilator circuit, (2) a small, portable sequencing device for real-time identification of pathogens based on their genetic material (metagenomic screening with the MINION® device from Oxford Nanopore Technologies, UK), and (3) a custom panel for quantifying biomarkers of host defense and lung injury that will be read using a small plate reader (FIG. 1). The portability and limited weight and power requirements of the system would facilitate use of these devices at Role 3 or 4 medical facilities.

Described herein is a 2-stage aerosol collector capable of providing independent sample collection from the distal lung. The inventors will use this collector to sample exhaled aerosols from ex vivo ventilated human lungs and human subjects with and without respiratory infections to determine detectability of DNA from bacteria, viruses, and fungi and biomarkers of lung injury and host response. The inventors will incorporate portable metagenomics technologies to analyze these samples and compare them to conventional lab-based assessments to establish that pathogens can be reliably and accurately identified in exhaled aerosols. The inventors will also quantify levels of host defense and lung injury biomarkers in exhaled aerosols. The inventors will determine the sampling time needed to obtain necessary aerosol sample volumes and compare aerosol samples to samples gathered through lung lavage.

### The Need for Rapid Detection of VAP and Identification of the Associated Pathogen:

There is an unmet need for more rapid and accurate diagnosis of VAP that affects civilian, Veteran's Administration (VA), and military medical systems. In a meta-analysis from the pre-COVID era, VAPs occurred in 5-40% of patients undergoing mechanical ventilation for at least 48 hours with 9% attributable mortality. Mechanically ventilated patients with traumatic injuries are disproportionately affected by high VAP rates. VAP has become even more common in the COVID era, with reported rates in range of 40-50% in mechanically ventilated COVID-19 patients. Diagnostic uncertainty around VAP often leads to indiscriminate, empiric antibiotic prescriptions with >85% of critically ill patients receiving antibiotics during their ICU course. Dedicated microbiologic work-up with gram staining and cultures of bronchoalveolar lavage fluid (BALF) collected via fiberoptic bronchoscopy can improve diagnostic yield and help better target antibiotics. Following diagnosis, VAPs have been associated with prolonged duration of ICU stay, longer time on mechanical ventilation, higher rates of failure to liberate from the ventilator, and excess all-cause mortality in the ICU.

Timely detection of VAP and correct identification of the pathogen is key to effective treatment. However, the diagnosis of VAP and the identification of the associated pathogen can be difficult. Clinical indicators of pneumonia, such as fever, leukocytosis, sputum production, or radiographic densities are non-specific (9) and may be due to non-VAP etiologies, including extrapulmonary infections, atelectasis, or pulmonary edema. Thus, to reach a VAP diagnosis, practitioners typically rely on microbiologic evidence of infection in the lower respiratory tract, i.e. they need a sample from the lungs for microbiologic testing. This diagnostic process has major limitations on both of its two necessary components: the sample type and the microbiologic testing.

The inventors thus rely on collection of lower respiratory tract secretions via the intubated airways to diagnose VAP. The most commonly used specimens are tracheal aspirates obtained via suctioning of distal tracheal secretions beyond the tip of endotracheal tube. Whereas tracheal aspirates are generally acceptable specimens for VAP diagnosis, they may not be representative of the infectious process in the distal parenchyma and can have limited specificity by detecting organisms colonizing the proximal airways. The current reference standard is bronchoscopy for collection of BALF, but this procedure is invasive, requires transient disconnecting of patients from ventilatory support with the potential for clinical decompensation and worsening of gas exchange, requires clinical operator expertise, and is not easily repeatable for surveillance. Consequently, the ability to sample the distal lung parenchyma in ways that are non-invasive, safe, reliable, and repeatable represents a critical unmet need in the care of mechanically ventilated patients in the ICU.

Regardless of the sample type, the next major hurdle in VAP diagnostics stems from inherent limitations of the conventional microbiologic methods currently in use. Microbiological diagnosis of VAP requires identification of pathogenic organisms that have grown above certain thresholds (in terms of colony forming units) on culture dishes. This process inherently requires time, which can vary substantially by organism, as fastidious bacteria or certain fungi can take several days or weeks to grow. This process is also inherently insensitive, as certain organisms cannot grow with commonly used protocols (by growth media, oxygen pressure or temperature parameters), whereas antecedent antibiotics may also inhibit the growth of otherwise easily cultured organisms. Finally, this process is also inherently non-specific, because organismal growth on an ex vivo petri dish does not necessarily prove causation, as even quantitative culture methods cannot reliably differentiate acute infection from harmless bacterial colonization.

These major limitations in the work-up of VAP translate into suboptimal therapeutic decisions. Antibiotics are routinely prescribed upon clinical suspicion of VAP, but microbiologic diagnosis may require several days to allow for tailoring the antimicrobials against the culprit pathogens. Thus, initial courses are empiric, and typically broad-spectrum to cover hospital-acquired pathogens with potential for antibiotic resistance. Such empiric antibiotics can be overtly broad (for patients without resistant pathogens), ineffective (for patients with extremely resistant pathogens or infections caused by unsuspected organisms), or even totally unnecessary (for patients without a true VAP). Such empiric courses are often continued when microbiologic cultures come back as negative for pathogens (for fear of missed pathogens due to low sensitivity by cultures) and the cumulative antibiotic exposure in the ICU drives antibiotic resistance, ablates indigenous microbiota predisposing patients to secondary infections (such as C. difficile colitis), and increases risk for toxicity and total cost. On the other hand, delays in diagnosing the causal pathogen(s) for patients with true VAP can limit access to effective therapies and may allow for progression of infection increasing the chances of lung injury, permanent impairment, or death.

Military ICUs in forward locations may experience higher rates of VAP. A 2007 study of patients at the Air Force theater hospital in Iraq reported VAP rates as high as 56/1000 ventilator days during different phases of operation—more than 5 times U.S. rates. In a study of wounded and evacuated military personnel during 2009-2010, 36/423 patients (9%) and 30/162 ICU patients (19%) developed pneumonias, 83% of which were VAPs. The associated pathogens varied and included gram-negative (56%) and gram-positive bacteria (18%) and fungi (also 18%), with 27% of pathogens being multi-drug resistant. There is the potential for deployed medical units to encounter novel environmental pathogens that may cause respiratory illness requiring mechanical ventilation, or new ICU pathogens that can lead to VAP. Such pathogens may be particularly difficult to identify, as they may not be included within routine microbiologic studies. There is previous history of such infections in the military health care system. Outbreaks of multidrug-resistant Acinetobacter baumannii-calcoaceticus occurred within the military healthcare system in Iraq. Melioidosis caused by Burkholderia pseudomallei was reported in Marines training in Australia. A series of 18 cases of eosinophilic pneumonia occurred amongst military personnel in Iraq in 2003-2004, with 12 patients requiring mechanical ventilation and two of them died, yet no etiology was clearly identified.

Cost Savings Associated with Early and Effective VAP Therapy:

The cost of a single VAP case was estimated to be over $47,000 in a AHRQ report from 2017. VAP was one of the two most costly hospital-acquired conditions described in the report along with central line associated bloodstream infections. Bundled prevention strategies have demonstrated efficacy in decreasing rates of VAP as well as the associated costs. An additional strategy for limiting the costs of these infections would be to treat them more effectively by providing targeted therapy earlier in the course of infection. Though this has not been specifically tested, targeted therapies would be expected to decrease time of mechanical ventilation and time in the ICU, both of which bear significant health care costs. A public price list from Cleveland Clinic from April 2022 listed the cost of a day of intensive care treatment as being $5,496. Mechanical ventilation may increase the cost of intensive care significantly. A recent study estimated that inappropriate empiric antibiotic therapy for bacterial VAP increased mechanical ventilation duration by 2 days, hospital length of stay by 3 days, and costs by 17%. At the same time, a highly sensitive diagnostic approach for VAP will also allow infection to be definitively ruled out safely, and thus allow for early termination of empiric antibiotics as well as for re-focusing the diagnostic work-up on alternative and likely untreated pathologies (such as atelectasis or cardiogenic pulmonary edema).

### Technology Element 1: Exhaled Aerosol Collection to Sample the Distal Lung

One aspect of the point of care system the inventors will develop is an aerosol collector that will allow the inventors to selectively sample secretions from the distal lung during mechanical ventilation. Exhaled aerosols have received only very limited consideration as a sample source for diagnosing respiratory disease despite their known role in conveying microbial pathogens.

Extracted fluids from heat and moisture exchangers (HME), sponge-like devices placed within the ventilator circuit to exchange heat and moisture between exhaled and inhaled air are a promising source for biomarker collection for assessing lung injury. These sponges effectively filter exhaled aerosols within mechanical ventilator circuits. Measurable levels of IL-10, IL-6, IL-8, TNF-α, MMP-9, MPO, RAGE, and SPD (amongst many other markers) were found in HME at concentrations similar to reference standard BALF providing evidence that exhaled aerosols carry content from the distal lung.

The advantages of the aerosol collector disclosed herein over HME sampling include higher efficiency aerosol collection which will provide measurable samples in less time allowing for frequent surveillance. The disclosed system can provide direct sampling of the distal lung fluid which avoids contamination by secretions from the upper airway that may confuse the diagnosis. The disclosed systems will not require the large centrifuge needed to extract fluid from HMEs. The disclosed systems will allow for aerosol sampling with any ventilator, filter, HME product in place.

Aerosols are generated in the proximal lung by high-speed airflows passing over secretions in the large airways during exhalation and in the distal lung by the rapid opening of small airways during inhalation. Aerosols generated in the distal lung will be smaller than those generated in the proximal lung because the lung filters out larger aerosols as they are exhaled through the airway tree. Aerosols generated in the proximal lung, throat, and mouth are subject to less filtering and therefore may be in larger sizes when exhaled. Exhaled aerosols from the distal lung are typically ˜1-10 μm in diameter while aerosols from the proximal lung may be much larger ˜100's μm. Collecting aerosols based on their size therefore, in principle, provides a means of selectively sampling the distal lung. The selectivity of this sampling along with the availability of microbial DNA and biomarkers within these aerosol samples will be established through the proposed research.

The inventors have developed a 2-stage prototype aerosol collector shown in the accompanying figures that has undergone preliminary testing. The first stage of the collector is designed to deposit large aerosols within an Eppendorf tube attached at the base of the stage. Smaller aerosols would pass into the second stage where they would be deposited in a second Eppendorf tube. Content in the first stage tube will include material from the large airways and condensate from within the vent line. Content with second stage tube will be from the distal lung. The impaction stages are housed within PVC pipe. Eppendorf tubes are contained within threaded holders that screw into each stage ensuring that the collector is airtight and able to maintain positive end-expiratory pressure (PEEP) when applied as part of mechanical ventilation. This will allow sampling without interference with the ventilator—a significant advantage vs. bronchoscopy.

### Aerosol Collector Prototype Preliminary Testing Data:

The inventors tested the collection efficiency of the device using two saline test aerosols delivered into the collector with a steady 20 L/min air flow from an air compressor. The inventors generated a small test aerosol using an Aerogen Solo electronic (vibrating mesh) medical nebulizer that had a volume median diameter of 4.3±0.1 μm and a large test aerosol using a syringe and nasal irrigation cannula which had a volume median diameter of 200±188 μm, as measured by laser diffraction (Malvern Mastersizer S). The inventors tested the aerosols separately. The inventors calculated the collection efficiencies of the impaction stages as the collected mass in the Eppendorf tubes divided by the starting mass of liquid in the syringe or nebulizer. The inventors also measured upstream back pressure of the collector device using a digital pressure gauge (Druck). The inventors demonstrated good collection efficiencies and specificity of collection by stage with large aerosols being collected primarily in the proximal lung stage and smaller aerosols being collected primarily in the distal lung stage of the device as shown in Table 1 below. Table 1 shows collection efficiencies assessed using test aerosols delivered into the collector in 20 L/min of constant air flow. Efficiency is % of saline volume loaded into the nebulizer (4 μm aerosol) or syringe (200 μm aerosol) that is recovered in the Eppendorf tube for each stage.

TABLE 1

| | | Distal | |
|---|---|---|---|
| | Proximal collection | collection | Back pressure |
| Test Aerosol | efficiency % | efficiency % | (cm H2O) |
| 4 μm | 0.1 ± 0.05% | 17 ± 2% | 3.3 ± 0.1 |
| (n = 6) | | | |
| 200 μm | 75 ± 13% | 1 ± 1% | 3.1 ± 0.2 |
| (n = 9) | | | |

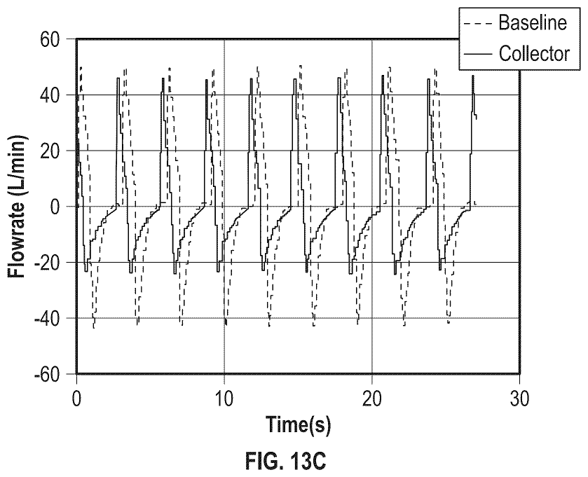

The inventors tested whether the collector would affect normal ventilator operation by placing it in the exhalation line of a mechanical ventilator circuit including a Puritan Bennett 980 series Ventilator and a Michigan Test lung. Sample tracings showing changes in test lung volume, airway pressure, and flowrate vs. time are shown in FIGS. 13A-13C with and without the collector. These results illustrate that the collector had only minor effects on tidal volume delivered and airway pressures but did decrease exhalation flowrates. As this effect might cause air to become trapped in the lungs at higher ventilation frequencies, the inventors are revising the design to decrease resistance within the collector and minimize air trapping. FIGS. 13A-13C illustrate traces from ventilator-test lung circuit comparing normal ventilator configuration (baseline) to the configuration with the aerosol collector placed in the exhalation line of the ventilator (collector). Measurements of volume (FIG. 13A), proximal airway pressure (FIG. 13B), and inhalation and exhalation flowrate (FIG. 13C) are shown. The collector had only minimal effects of volume and pressure.

The inventors received IRB approval to collect exhaled aerosol samples from healthy subjects who exhaled through the collector for a period of 45-60 minutes. The inventors collected samples from the first enrolled subject who exhaled through the device during 5 sessions. Proximal and distal aerosol samples were collected. Table 2 shows the average mass of aerosol collected from each stage on a per session and per minute basis where a healthy volunteer exhaled through the device for 45-60 minutes. Mean of 5 sessions is presented ±SD. These results illustrate that the system effectively collects aerosol samples during exhalation. Variability in the proximal airway sample mass is likely due to variable levels of contamination by condensate within this sample. Mass collected within the distal lung stage was consistent, indicating that the current system can be used to investigate the content of the exhaled aerosols while the next generation prototype is being developed.

TABLE 2

| Aerosol Collection | Average Sample mass | Average Sample mass |
|---|---|---|
| Stage | collected/session (mg) | collected per minute (mg) |
| A-proximal | 237 ± 213 | 4 ± 4 |
| B-distal | 52 ± 17 | 0.9 ± 0.2 |

### Technology Element 2: Metagenomics for Detection and Identification of Pathogens

The second element of the point of care system the inventors propose to develop is a portable metagenomics technology, which will be used to analyze the aerosol samples collected from mechanically ventilated patients. These technologies use real-time metagenomic sequencing of all DNA molecules in a sample to comprehensively screen for and then identify and quantify the abundance of sequences belonging to pathogens. In certain examples, existing technologies can be utilized for this purpose their applicability tested for the disclosed systems. An example of an existing point of care metagenomic technology is the MINION® Mk1c device by Oxford Nanopore Technologies. The device weighs less that 450 g and would be easily portable. It can detect in real-time bacterial, viral, and fungal pathogens based on their genetic material (either DNA or RNA, depending on used protocols). Here the inventors will first determine the sensitivity and specificity of this technology for detecting and identifying DNA pathogens using exhaled aerosol samples, as DNA organisms account for most of known VAP cases. The inventors will compare bacterial community classifications generated by Nanopore metagenomics with the MINION® Mk1c device against two benchmark methods: i) traditional microbiologic cultures as routinely performed by the hospital laboratory and ii) culture-independent sequencing of bacterial and fungal marker genes, as extensively done by the inventors. The marker gene sequencing approaches (16S rRNA gene for bacteria and ITS [Internal transcribed spacer] for fungi) are sensitive methods that amplify the target gene and perform massive parallel sequencing of multiple pooled samples at the same time, with well-established experimental and analytical pipelines. The inventors will use a series of aerosol samples from an ex vivo lung model, filters collected from ventilators in ICUs, and studies with the aerosol collector designed in AIM 1 involving healthy participants and participants with symptoms of lung infections. Below the inventors describe their experience in performing large-scale investigations of the lung microbiome and present preliminary data describing the use of 16S technology to assess exhaled aerosol samples for bacterial DNA.

Preliminary Studies with Metagenomic Technologies:

Metagenomic techniques offer great advantages over marker gene sequencing approaches, such as 16S and ITS. Metagenomics can sequence longer DNA molecules, providing improved taxonomic resolution (i.e. identification of species or strains, and not just crude genera as done by 16S), they can sequence DNA (or RNA) from all organisms in a sample, thus capturing bacteria, viruses and fungi in a single experiment), and can also detect the presence of antibiotic resistance genes, which can have direct implications for care. With regards to clinical applicability, the main advantage of Nanopore MINION® (Oxford Nanopore Technologies, UK) is the rapidity and versatility of its protocols which allow for real-time data acquisition in clinically actionable timelines of 5-6 hrs from sample to results. Nanopore sequencing can be performed with small, portable, and affordable devices with the potential for point of care applications.

### Technology Element 3: Biomarkers to Assess the Extent of Infection and Lung Injury

Metagenomic sequencing methods can be highly sensitive to pathogen detection in lower respiratory tract samples. However, various microbes, including typical pathogens, can be detected within the lung even in the absence of acute infection. Such organisms could be normal community members (i.e. commensals) or quiescent pathogens that colonize the airways but do not cause invasive infection at that time-point. Although metagenomics can provide measures indicative of the presence or absence of infection (such as dominance of a community by a typical pathogen vs. a highly diverse community of commensals), they do not inform on the impact of microbes on the host. Contemporary understanding of pneumonia conceptualizes the disease as “a specific inflammatory response to a specific bacterial community dominated by a specific, niche-adapted pathogen”, Therefore, to reliably diagnose VAP, the inventors not only need sensitive methods to profile microbes; the inventors need to simultaneously profile the host responses to microbes and synthesize these two components into an integrative diagnosis. Therefore, the inventors will incorporate a panel of host defense biomarkers that will indicate the extent to which the immune system is responding to the infection. These biomarkers will be assessed in both exhaled aerosol and plasma. The inventors anticipate that the exhaled aerosol will provide a better indication compared to plasma samples because the aerosol sample is collected directly from the site of infection. If fully realized through this research, a series of biomarkers could be then assembled into an ELISA panel with a simple plate reader as a point of care tool for quantifying host response using exhaled aerosol samples.

The inventors will also assess biomarkers that provide an indication of the extent of lung injury. These biomarkers will be useful for performing further research on the evolution of lung injury and acute respiratory distress syndrome (ARDS) and may be useful for guiding future ARDS treatments. Recent studies have shown that plasma biomarkers of inflammation (ST2 and IL-6) are useful for quantifying the extent of lung injury and predicting successful ventilator liberation.

Exhaled aerosols may provide a more direct and concentrated source of biomarkers than plasma, facilitating detection earlier in the disease course. Extracted fluids from heat and moisture exchangers (HME)—sponge-like devices placed within the ventilator circuit to exchange heat and moisture between exhaled and inhaled air have been shown to be a promising source for ARDS-related biomarker collection. Measurable levels of IL-10, IL-6, IL-8, TNF-α, MMP-9, MPO, RAGE, and surfactant protein D (SPD) (amongst many other markers) were found in HME at concentrations similar to edema fluid. This provides proof of concept that relevant biomarkers are recoverable from exhaled aerosols.

### Hypotheses:

Described herein is a highly efficient, non-invasive, point of care system for collecting and analyzing exhaled aerosols from the lungs of mechanically ventilated patients that will provide rapid detection and identification of viral, bacterial, or fungal pathogens. The system can also include measurements of biomarkers to determine the extent of infection and lung injury. Such a system would allow for early diagnosis and treatment of lung infection that could prevent lung injury, loss of respiratory function, and death.

The systems and devices described can include three technologies that can be part of the point of care system: (1) a small passive aerosol collection device that can be placed in the exhalation line of the ventilator, (2) an available metagenomics technology used to identify pathogens, and (3) biomarker analysis for determining the extent of infection and lung injury. The systems and devices described herein can provide the following features and/or advantages.

1: A highly selective exhaled aerosol collector can be developed with a distal stage that will collect fluid originating from alveoli and smallest airways with less than 5% contamination from the large airways, as demonstrated through ex vivo human lung experiments.

2: Microbial community assessments from distal lung exhaled aerosol samples collected over a 60-minute period can be similar to assessments from bronchoalveolar lavage as demonstrated through ex vivo human lung experiments.

3: Metagenomic identification of microbial community members can show good agreement with culture-based methods and 16S/ITS sequencing methods in samples from: (1) exhaled aerosol collected from the ex vivo lung model, (2) discarded ventilator filters from mechanically ventilated patients, (3) exhaled aerosol collected from healthy study controls, (4) exhaled aerosol from cystic fibrosis (CF) patients with symptoms of lung infection.

4: Inflammatory biomarkers can be detectable in exhaled aerosol samples and can increase with severity of infection and with ARDS.

### Specific Aims:

Specific Aim 1: To develop and optimize a highly efficient system for collecting exhaled aerosols from the lungs of mechanically ventilated patients and test its function in a ventilator circuit.

Specific Aim 2: To compare microbial community assessments from exhaled aerosols to bronchoalveolar lavage and determine the aerosol sampling time required to provide accurate pathogen detection/identification.

Specific Aim 3: To compare point of care metagenomic methods for identifying bacterial, fungal, and viral pathogens to standard culture-based and marker gene sequencing-based methods using exhaled aerosol samples collected from an ex vivo lung model, discarded ventilator filters, and healthy and infected human subjects.

Specific Aim 4: To determine whether biomarkers associated with inflammation, epithelial and endothelial dysfunction, and lung permeability are altered in samples of exhaled aerosols from patients with pulmonary infections.

### Research Strategy:

Specific Aim 1: To develop and optimize a highly efficient system for collecting exhaled aerosols from the lungs of mechanically ventilated patients and test its function in a ventilator circuit.

Primary design features can include: (1) limited resistance to prevent interference with ventilator function, (2) general ventilator compatibility under a variety of typical ventilator operating conditions, (3) high aerosol collection efficiency to facilitate timely surveillance, (4) high selectivity for collecting aerosols from the distal lung in stage 2 of the device.

Stage 2 collects aerosol by directing exhalation flows through a short nozzle that directly impacts the air and aerosol onto the internal surfaces of the Eppendorf tube. This direct collection method was intended to minimize sample losses and does result in relatively high collection efficiencies (see Table 1). However, it can be associated with airflow resistance that may affect exhalation flowrates during ventilator testing, which the inventors believe can be alleviated through a redesign of the stage as shown in, for example, FIGS. 3A and 3B. This design includes a spherical impaction surface that can freely rotate on a shaft transferring deposited liquid into a funnel positioned directly above the Eppendorf tube. Air flow will cause rotation of the sphere contributing to liquid transfer to the funnel and into the Eppendorf. The design allows for the use of a less constrictive nozzle and instead effects impaction through proximity between the wider nozzle and the impaction sphere. Impaction efficiency of the stage can be proportional to Stokes number: Stokes=ρd²V/18μD where ρ is air density, d is aerosol size, V is air velocity, μ is air viscosity, and D is a geometric parameter. In the design of FIG. 3A the inventors speculate that D is the difference in width between the nozzle and the Eppendorf. In the design of FIG. 3B the inventors speculate that D is the distance from the nozzle to the impaction sphere.

Aerosol collector bench testing: The inventors will test the aerosol collection efficiency of the updated design on the bench using two different test aerosols and continuous airflow. This testing process will also be used to optimize the design—for example determining the gap between the nozzle and impaction sphere that provides the best combination of high collection efficiency and low resistance. A small test aerosol will be generated using an Aerogen Solo electronic (vibrating mesh) medical nebulizer (volume median=4.3±0.1 μm). A large test aerosol will be generated using a syringe and nasal irrigation cannula (volume median=200±188 μm). A continuous 20 L/min airflow will be used to convey the aerosol through the collector during testing. Aerosol collection efficiency for each stage will be measured as the mass collected in the Eppendorf tube divided by the total mass added to the nebulizer or syringe. Resistance will be assessed by measuring the back pressure associated with the collector. The inventors will repeat each test case 3 times. The exact number of test cases will depend on the evolution of the design. The inventors' design goal is to have 20% efficient collection of small aerosols in the distal lung stage, 95% efficient collection of large aerosols in the proximal lung stage, and back pressures from the collector similar to those associated with ventilator filters (approximately 2.3 cm H2O at 60 L/min, based on Gibeck HEPA Lite filter).

Aerosol collector ventilator testing for patient safety: The inventors will place the aerosol collector in line with a mechanical ventilator and a Michigan test lung and assess the effect of the collector on ventilator performance. Measurements will include time traces of delivered air volume, proximal airway pressure, and inhalation and exhalation flow rate. These will be compared for each test with and without the aerosol collector in place. The inventors will use a Puritan Bennett 980 Ventilator. The inventors will vary airway resistance and lung compliance on the test lung. Compliance: 0.04-0.10 L/cmH₂O. Resistance: 5 to 20 cmH₂O/s. The inventors will vary the following parameters related to ventilator operation: tidal volume, mode, respiratory rate, positive end expiratory pressure (PEEP), and the presence of spontaneous breathing (available through an attachment for the test lung). Testing protocols will be guided by physician investigators who regularly care for mechanically ventilated patients in the intensive care units: One or more prototypes will be tested during 30 minutes of ventilator operation, under 5-10 test conditions (combinations of ventilator and test lung settings). Successful testing will indicate similarly between ventilator performance with and without the collector within 15% of expected parameter values, and absent of effects that could be harmful to the patient such as “breath stacking”—a consistent increase in lung volumes associated with incomplete exhalation.

### Ex Vivo Lung Testing:

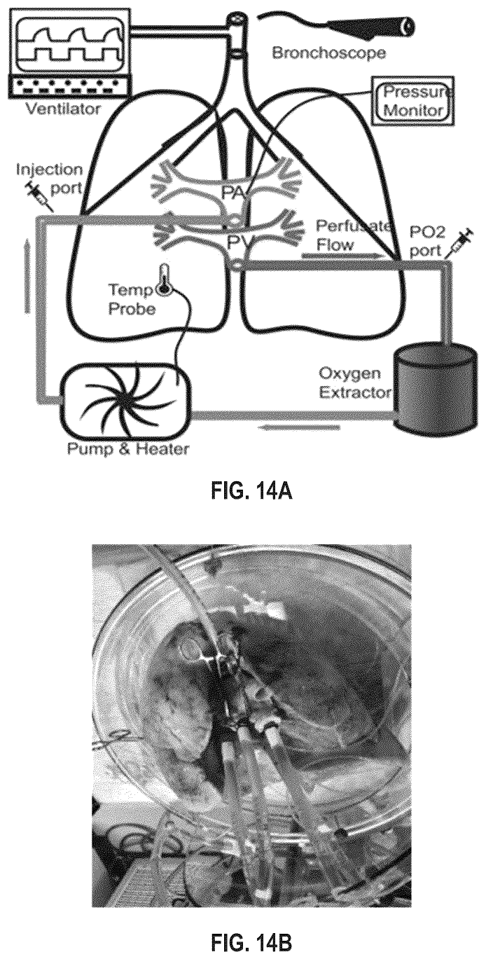

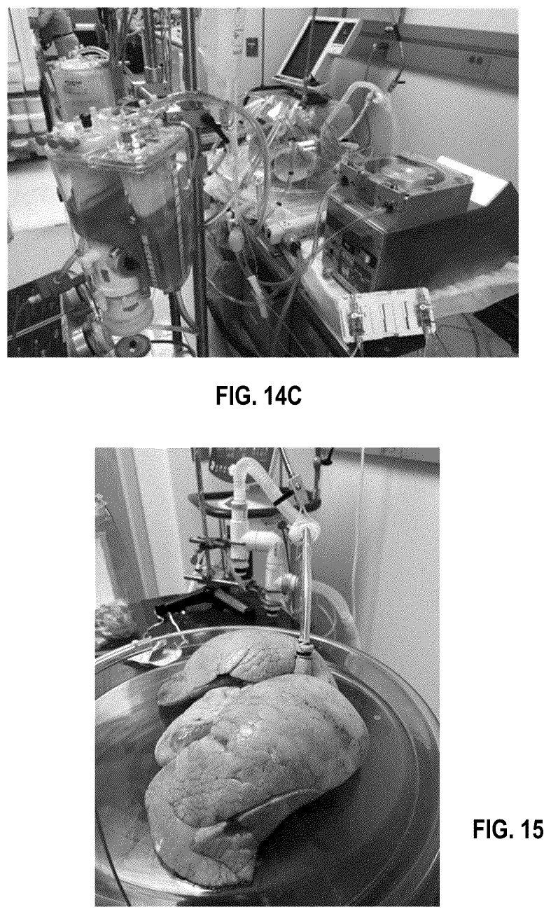

The inventors will utilize an ex vivo human lung model to validate the selectivity of distal lung aerosol collection and to compare sampling by aerosol collection to sampling by bronchoalveolar lavage. This model is shown in FIGS. 14A-14C and FIG. 15. FIG. 14A is a diagram illustrating lungs and a ventilator, and FIGS. 14B and 14C are images of the perfused and ventilated ex vivo human lung model to be used in these studies. FIG. 15 illustrates the sample collection apparatus of FIG. 6 undergoing testing and coupled between a mechanical ventilator and a pair of ex vivo lungs. The lungs used in these experiments will be obtained for transplant but found to be unusable when they arrive at the center thus entering research protocols. The inventors have previous experience with this model.

The inventors will add a small amount (10 mCi) of Technetium 99m pertechnetate added to the perfusate (MW=186 Da). This probe is small enough to diffuse from the capillary bed into the alveoli. The inventors anticipate that this fluid will reach the lumen of the smallest airways and be aerosolized. If the collector is selective for aerosols generated in the distal lung, then this radioactivity should be recovered in the distal stage of the collector. The inventors will allow the radiolabeled perfusate to circulate for 30 minutes while the lung is ventilated prior to the start of the experiment. The bronchial circulation will not be perfused and therefore the probe should not be introduced directly into the airways. During that time will use a bronchoscope to spot fluorescently labeled (70 kDa) dextran dye onto different portions of the airway tree. These spots will be approximately 5 mm-1 cm in diameter. Three spots will be placed in the trachea, one in each mainstem, and one spot in each lobar bronchus being careful to avoid any distal delivery of the dye. The inventors will collect exhaled aerosol while the ex vivo lungs are ventilated over a period of 6 hours. Eppendorf tubes will be changed out once per hour in the distal and proximal collector stages. Sample radioactivity and fluorescence will be assessed in each. Fluorescence associated with the dye (as measured by spectrofluorimetry) will be indicative of material originating from the airways. Radioactivity associated with the Technetium 99m will be associated with material originating from the alveoli and the respiratory and terminal bronchioles in communication with the alveoli. Finally, at the conclusion of the experiment the inventors will perform bronchoalveolar lavage (BAL) by inserting a bronchoscope through the trachea and washing fluid into the distal lung and then collecting it by suction. This is the same process that is used clinically to collect samples from the distal lung for pathogen identification.

Hypothesis 1: A highly selective exhaled aerosol collector can be developed with a distal stage that will collect fluid originating from alveoli and smallest airways with less than 5% contamination from the large airways as demonstrated through ex vivo human lung experiments

The inventors anticipate that concentration of radioactivity in the distal lung sample will match concentration of radioactive counts/ml in the perfusate as the small molecule probe will have diffused from the capillary bed into the thin liquid layer lining the alveoli and smallest airways. The inventors anticipate only minimal dye will be found in this sample, specifically that dye concentration in the distal collection stage will be 5% or less than the concentration deposited in the airway tree. The inventors anticipate that larger aerosol droplets containing dye will be collected in the proximal collector and that dye concentration in the proximal sample will 20-50× higher than the distal collector. The inventors anticipate that radioactivity concentration in the proximal stage will be minimal (˜5% of the distal collector). This experiment will be repeated 3 times.

Specific Aim 2: To compare microbial community assessments from exhaled aerosols to bronchoalveolar lavage and determine the aerosol sampling time required to provide accurate pathogen detection/identification.

The inventors will assess microbial communities in the exhaled aerosol samples from the ex vivo lung model using 16S sequencing for bacteria and ITS sequencing for fungi, and compare them to similar assessments in BAL. This will allow the inventors to determine whether 60 minutes is a sufficient time for aerosol collection and whether community assessments in exhaled aerosol are similar to BAL which is the current gold standard sampling method for the distal lung. In addition to the 3 ex vivo lung studies performed in Aim 1, the inventors will perform an additional 3 ex vivo studies for Aim 2 that will include aerosol collection and BAL but will not include radiolabeling of the perfusate or instillation of dye into the airways.

16S rRNA Gene and Internal Transcribed Spacer (ITS) Region Sequencing. The inventors will use approaches as described in the analysis of lung microbiome from aerosol samples section. Since the inventors anticipate low microbial biomass samples, special care will be taken to avoid the introduction of exogenous DNA and to measure potential background contaminants. For 16S and ITS sequencing, DNA will be PCR amplified in triplicate (to ensure rigor and reproducibility). PCR master mixes will be treated with nucleases to reduce potential background from contaminants (ArcticZymes Technologies, Norway). Inline indexed (barcoded) V4 16S primers will be used for bacterial amplification and fungal species will be interrogated using sequences derived from the ITS region in an analogous fashion using primers targeting the ITS2 region (36, 43, 44). For data rigor and reproducibility, each experimental step (DNA extraction and PCR) will include reagent blanks as negative controls, and mock community standard (ZymoBIOMICS™, Zymo Research) as positive controls. Amplicons will be cleaned, normalized, and pooled. Library concentrations will be calculated, normalized, pooled, and run on the Illumina Miseq platform with v3 (600 cycle) kits. Sequencing will be completed to generate at least 10,000 high quality reads per sample.

16S and ITS Taxonomic Profiles. The 16S and ITS reads will be processed with the inventors' standard in-house quality control (QC) pipeline as described in the preliminary data. ITS reads will be classified using the UNITE ITS database. The pipeline also generates Operational Taxonomic Units (OTUs). Results from this 16S/ITS pipeline will be converted into per sample taxonomic profiles represented as categorical counts in matrices of dimension (number of samples)×(number of categories) for subsequent use in analyses described below.

Environmental Controls: At each time point, samples will be taken to measure exogenous background contamination. From BAL samples, control sample (saline flushed through the bronchoscope) will also be obtained. A similar process will be performed with the catheters used to sample endotracheal aspirate. Taxonomic distribution patterns from the environmental controls will be used to identify contaminants in the experimental samples by fitting a mixture model. The mixture model assumes that the sequenced observed sample is a combination of a hypothetical clean (no exogenous contamination) and the empirically determined environmental control sample, at a proportion that is unknown but can be computed with an optimization algorithm.

Droplet Digital PCR (ddPCR); The inventors will measure microbial burden (Domain Bacteria and Fungi) using droplet digital PCR (ddPCR)(48). ddPCR is a form of quantitative PCR based on water-emulsion droplet technology that facilitates absolute quantification of nucleic acids with high analytical sensitivity and precision. ddPCR fractionates PCR reagents into tens of thousands of nanoliter or picoliter reactions using microfluidics. Each droplet contains 0 or 1 DNA template. The target nucleic acids are calculated by Poisson statistics from the number of positive droplets to provide precise, absolute target quantification.

Hypothesis 2: Microbial community assessments from distal lung exhaled aerosol samples collected over a 60-minute period will be similar to assessments from bronchoalveolar lavage as demonstrated through ex vivo human lung experiments

For Hypotheses 2, comparisons will be performed using distance-based, abundance-based, and distribution-based approaches where each method accounts for the compositional nature of the sequence data and provides opportunities to integrate and determine associations with other experimental variables through statistical models. The inventors' primary focus will be on the comparison between exhaled aerosol samples and BAL collected from (n=6) ex vivo lung models. As these are matched samples (from the same lungs), paired difference analyses will be performed to examine the degree of differences between the two samples methods (BAL and aerosol) from the same subject by computing the difference (delta) in taxonomic abundance, overall composition, or diversity between the two samples from the same lung that can be incorporated into linear models. The inventors anticipate good agreement between the samples.

Specific Aim 3: To compare point of care metagenomic methods for identifying bacterial, fungal, and viral pathogens to standard culture-based and marker gene sequencing-based methods using exhaled aerosol samples collected from an ex vivo lung model, discarded ventilator filters, and healthy and infected human subjects.

Metagenomics methods: Human DNA is highly abundant in clinical samples and can overwhelm the sequencing output of metagenomic technologies that agnostically screen all DNA molecules. Therefore, the inventors will apply a human DNA depletion step when processing samples using a detergent-based method to lyse human cells followed by digestion of human (non-bacterial) DNA with nuclease. The inventors will then extract Genomic DNA using the DNeasy PowerSoil kit (Qiagen). The inventors will prepare metagenomic sequencing libraries with a Rapid PCR Barcoding Kit (SQK-RPB004) and then perform sequencing on the MinION Mk1C device [Oxford Nanopore Technologies—ONT, UK]. Run time will be 5 hours. The inventors will process the output with the Guppy software and the EPI2ME platform from ONT for quality control, species identification [What's In My Pot (WIMP) pipeline] and antimicrobial resistance gene analyses [ARMA workflow]. The inventors will exclude samples that generate fewer than 300 high-quality microbial reads from further analyses. For internal quality control of the reliability and reproducibility of Nanopore sequencing, the inventors will perform also include positive control samples with extracted DNA from a mock microbial community with known composition (ZymoBIOMICS Microbial Community Standard) and compare derived vs. expected abundance of microbial species.

Aerosol collection from human subjects: The inventors will enroll two groups of human subjects who will provide exhaled aerosol samples by breathing through the collector. These subjects will not be mechanically ventilated. The first group (n=12) will include healthy volunteers ages 18 and older who do not smoke or vape and have no known lung disease The second group will include 12 people with cystic fibrosis (CF) ages 18 and older who have symptoms of a respiratory infection. (CF patients are prone to pulmonary infection and lung inflammation and thus this group provides a disease control for studies.) All participants will exhale through the collector for 30 minutes. The inventors will also collect oral wash and expectorated sputum (if possible, without induction) and blood. Samples will be processed for metagenomics, bacterial community identification via 16S sequencing, fungal identification via ITS sequencing, and biomarker assessment (aerosol and plasma, described in Aim 4). The inventors will obtain permission to follow the medical records of the CF group to see if a pathogen is identified. Since the inventors' methods are experimental, they will not be applied for the care of the patient.

Hypothesis 3: Metagenomic identification of microbial community members will show good agreement with culture-based methods and 16S/ITS sequencing methods in samples from: (1) exhaled aerosol collected from the ex vivo lung model (n=6), (2) discarded ventilator filters from mechanically ventilated patients (n=30), (3) exhaled aerosol collected from healthy study controls (n=12), (4) exhaled aerosol from cystic fibrosis (CF) patients with symptoms of lung infection (n=12).

The inventors will use the 16S and ITS microbiota profiles from amplicon sequencing as the expected outcome and the metagenomics bacterial and fungal profiles as the observed outcome for statistical comparison. This approach will allow the inventors to determine the overlap between the two methods and what taxa may be unique to one method. The inventors will also compare metagenomic outcomes with culture-based outcomes and anticipate dominant culture detected microbes will have demonstrate high rates of abundance through metagenomic analysis based on previous studies.

Specific Aim 4: To determine whether biomarkers associated with inflammation, epithelial and endothelial dysfunction, and lung permeability are altered in samples of exhaled aerosols from patients with pulmonary infections.

Measurements of biomarkers in exhaled aerosol samples indicating host response or altered lung permeability may be useful for interpreting metagenomic outcomes, providing specific insights as to whether detected pathogens represent acute infection or harmless colorizations. Here the inventors will analyze samples collected in the previous aims to measure levels of candidate biomarkers. This will allow the inventors to determine biomarker detectability in exhaled aerosols, compare levels vs. BAL, determine baseline levels in samples from healthy subjects, and alterations associated with pulmonary infection or ARDS. The inventors will also analyze biomarkers in collected plasma.

Indications that biomarkers will be detectable are available from previous studies where fluids extracted from ventilator heat and moisture exchangers contained measurable levels of IL-13, IL-6, IL-8, TNF-α, MMP-9, MPO, RAGE, and surfactant protein D (SPD) (amongst many other markers) at concentrations similar to edema fluid. These exchangers are positioned downstream of exhalation flows from the patient and are a collection point for exhaled aerosols.

Our group has previously described a system for phenotyping patients with acute respiratory failure based on plasma biomarkers as well as more recently with tracheal aspirate. Here the inventors will measure the level of a series of biomarkers including albumin (lung permeability), RAGE, SPD (epithelial dysfunction), ANG-2 (endothelial dysfunction), IL-6, IL-8, IL-10, TNFR1, ST2, GDF15 (inflammation), and procalcitonin/pentraxin-3 (bacterial infection) in samples collected from the experiments described in the previous aims, including: exhaled aerosol and bronchoalveolar lavage from the ex vivo lung model, samples collected from discarded ventilator filters from mechanically ventilated patients, exhaled aerosol from healthy study controls, and exhaled aerosol from CF patients with symptoms of lung infection.

The inventors will compare biomarker levels in exhaled aerosols vs BAL and plasma. The inventors will assess whether a 60-minute sampling period for aerosol collection during mechanical ventilation is sufficient to allow for quantification of baseline biomarker levels. Using samples from ventilator filters from patients with and without known infection or ARDS and exhaled aerosol samples from healthy participants and participants with cystic fibrosis and symptoms of lung infection, the inventors assess biomarker detectability and changes in biomarker response to pulmonary infection and ARDS. Biomarker data can also be compared to metagenomic assessments such as relative abundance to determine whether there are any significant correlations.

The combination of a highly efficient aerosol sampler and methods for assessing the biomarkers of lung injury will also allow for detailed studies of the temporal evolution of lung injury. Currently there are no methods available to collect frequent repeated samples from the distal lung. Bronchoalveolar lavage is to too invasive to be repeat frequently. Biomarker collection from heat and moisture exchangers/ventilator filters is promising, but the inventors propose that out collector will provide viable samples in less time. This would allow for more detailed assessment of the temporal evolution of lung injury, repair, and resolution.

Hypothesis 4: Inflammatory biomarkers will be detectable in exhaled aerosol samples and will increase with severity of infection and with ARDS.

The inventors will assess detectability of baseline levels using samples from non-infected subjects. These will be available from the ex vivo lung studies (n=6), aerosol collected from healthy participants (n=12), and ventilator filters from subjects without indications of pneumonia (n˜15).

### Alternate Approaches:

It is notable that metagenomic technologies are evolving rapidly, and that novel technologies could be utilized within the framework proposed here very easily. The inventors will consider incorporating these as they become available. The inventors will consider whether a system can be devised using 16S/ITS sequencing technologies with a minimal footprint and time requirements for these purposes. The inventors will also consider the use of targeted panels for pathogens such as the BIOFIRE® available from BioFire Diagnostics.

### Example 5: Additional Research and Applications of Impactor Sample Collection Apparatus

Ventilator associated pneumonias (VAP) are a common complication in mechanically ventilated patients, occurring more than 250,000 times annually. They occur in a high percentage of patients with acute respiratory distress syndrome (ARDS, 29%) and COVID-19 (>50%). Timely detection and identification of the VAP pathogen is key to effective treatment, as is determining whether positive cultures indicate acute infection or harmless colonization. The most common options for microbiological diagnosis are cultures of endotracheal aspirate or bronchoalveolar lavage (BAL). Endotracheal aspirate can be sampled non-invasively but does not directly indicate conditions in the distal lung and is not highly specific for diagnosing VAP. BAL directly samples the distal lung but is invasive and cannot be repeated frequently for surveillance.

Described herein is a non-invasive, high-efficiency system for collecting exhaled respiratory aerosols from mechanically ventilated patients. This system will collect large aerosols, typically generated in the proximal airways, separately from smaller aerosols, which are typically generated in the distal lung, thus providing independent samples from these two distinct compartments. The collection of a sample from the distal lung will facilitate the correct diagnosis VAP. The system will allow for short sample collection times, facilitating repeated measures and timely surveillance, and will be developed and tested in an ex vivo human lung model using culture and non-culture-based methods.

The inventors will also evaluate the utility of respiratory aerosol samples for surveying the microbial and fungal community members in the lung (the lung microbiome). Misbalances in the bacterial communities (respiratory dysbiosis) have been associated with decreased survival in mechanically ventilated patients and may play a role in both local and systemic inflammation. The inventors will use 16S rRNA gene and Internal Transcribed Spacer (ITS) region sequencing to identify bacterial and fungal community members and trial metagenomic methods.

Finally, the inventors will evaluate the use of exhaled aerosol samples to quantify host response to lung injury through the analysis of biomarkers related to inflammation and endothelial and epithelial permeability. Markers of host response and lung injury may help to differentiate bacterial colonization from acute infection and VAP. Methods for assessing the extent of lung injury may be useful for clinical decision making. They may also be useful for studying the temporal evolution of, and recovery from, lung injury and ARDS.

Aim 1: To develop a highly efficient device for collecting exhaled aerosols from the lungs of mechanically ventilated patients. The inventors will design a two-stage impaction collection system that fits into the exhalation line of the ventilator circuit and collects proximal and distal lung samples directly into Eppendorf tubes. The device will not interfere with ventilator performance and will allow for sample collection without opening the ventilator circuit. The inventors will use an ex vivo human lung model, radiolabeled perfusate, and fluorescently labeled airway secretions to validate the independence of proximal and distal lung samples.

Aim 2: To compare quantitative assessments of the bacterial and fungal communities in distal exhaled aerosol to BAL in ex vivo lungs. The inventors will collect samples of exhaled aerosol from ventilated ex vivo lungs prior to performing BAL. The inventors will perform quantitative assays of the bacterial and fungal communities found in the proximal and distal lung aerosol samples and compare them to the BAL and the endotracheal aspirate. Studies will include healthy and explanted COPD, IPF (Idiopathic Pulmonary Fibrosis), and CF lungs.

Aim 3: To measure temporal changes in biomarkers of host response in exhaled aerosol samples collected from an ex vivo human lung model during an LPS induced lung injury.

Supplementing pathogen detection with measures of host response will help to differentiate acute VAP infection from colonization. Measures of inflammatory cytokines along with indicators of increased endothelial or epithelial permeability may also be useful for determining the extent of lung injury and studying the temporal evolution of lung injury and ARDS. The inventors will collect serial samples of exhaled aerosol before, during, and after LPS is infused into to the perfusate of a perfused and ventilated ex vivo lung model and assess the concentration of potential biomarkers. Bronchoalveolar lavage (BAL) will be performed at the end of the experiment and biomarker levels in BAL will be compared to aerosol. Candidate biomarkers include albumin (permeability), RAGE, SPD (epithelial dysfunction), ANG-2 (endothelial dysfunction), IL-6, IL-8, IL-10, TNFR1, ST2, GDF15 (inflammation), and procalcitonin/pentraxin-3 (bacterial infection).

### A) Significance:

The long-term goal of this project is to develop a highly efficient, non-invasive system for collecting and analyzing exhaled aerosols from the lungs of mechanically ventilated patients that will allow for one or more of the following:

### [1] Rapid Detection of Ventilator Associated Pneumonia (VAP)

In a meta-analysis from the pre-COVID era, VAP occurred in 5-40% of patients treated with mechanical ventilation for at least 48 hours with 9% attributable mortality. Higher rates were reported in trauma and cancer. Recent studies report VAP rates of over 50% in mechanically ventilated COVID-19 patients.

VAP can be difficult to diagnose. Clinical indications of VAP such as fever or radiographic densities are non-specific and may be associated other common causes such as extrapulmonary infection and pulmonary edema, necessitating microbiological diagnosis. Samples of endotracheal aspirate can be collected non-invasively for culture but have limited specificity and may not represent conditions in the distal lung. Bronchoalveolar lavage (BAL) allows for sampling of the distal lung but is invasive and cannot be repeated frequently for surveillance. Regardless of the sample type, the culture-based methods currently used to diagnose VAP require time for growth to provide a diagnosis, are limited by decreased sensitivity after antibiotic use, and cannot easily detect some pathogens such as anaerobes and fungi. Quantitative methods applied in culture-based systems also often cannot differentiate acute infection from harmless bacterial colonization.

VAPs have been associated with prolonged length of stay in the ICU and in the hospital, increased hospitalization costs, longer time on mechanical ventilation, higher rates of failure to liberate from the ventilator, and excess all-cause mortality in the ICU. Timely detection and identification of the pathogen is key to effective treatment of VAPs, as is assessment of whether positive cultures indicate acute infection or harmless colonization.

A high-efficiency aerosol collector coupled with 16S rRNA-based methods can allow frequent non-invasive sampling for timely VAP surveillance. Independent sampling of the proximal and distal airway would help to differentiate acute infection from colonization, as would biomarkers of host response (AIM 3).

[2] Measurements of Host Response Biomarkers to Aid in the Diagnosis of VAP, Assess Lung Injury, and Facilitate the Study of Lung Injury and ARDS. Markers of host response and lung injury may help to differentiate bacterial colonization from acute VAP infection. Bacterial colonization is unlikely to generate a robust immune response while acute infection is likely to increase inflammatory cytokines and other indicators of bacterial host response that may be detectable in exhaled aerosols. Measures of inflammatory cytokines along with indicators of increased endothelial or epithelial permeability may also be useful for determining the extent of lung injury and studying the temporal evolution of lung injury and ARDS. Recent studies have shown that plasma biomarkers of inflammation (ST2 and IL-6) are useful for quantifying the extent of lung injury and predicting successful ventilator liberation. Exhaled aerosols may provide a more direct and concentrated source of biomarkers than plasma, facilitating more rapid detection, more accurate quantification, and possibly a wider array of measurable biomarkers. Extracted fluids from heat and moisture exchangers (HME)—sponge-like devices placed within the ventilator circuit to exchange heat and moisture between exhaled and inhaled air have been shown to be a promising source for ARDS-related biomarker collection. Measurable levels of IL-10, IL-6, IL-8, TNF-α, MMP-9, MPO, RAGE, and surfactant protein D (SPD) (amongst many other markers) were found in HME at concentrations similar to edema fluid. This provides proof of concept that relevant biomarkers are recoverable from exhaled aerosols. Advantages of the aerosol collector described herein dover HME sampling include higher sampling efficiency, which will provide measurable samples in less time, allowing for frequent VAP surveillance, and independent collection of proximal and distal lung samples based on size selection of the exhaled aerosol. This will allow for more specific assessment of conditions in the deep lung.

### B) Aerosol Collection Devices:

A device for collecting exhaled aerosols from mechanically ventilated patients that: (i) is highly efficient and able to provide analyzable samples in a minimal time, facilitating use for VAP surveillance. (ii) adds minimal resistance to exhaled flows, (iii) does not interfere with vent operation and allows for sample collection without opening the vent circuit. (iv) collects large aerosols from the proximal lung airways separately from small aerosols from the distal lung, (v) collects samples directly into Eppendorf tubes to minimize sample loss.

Ex vivo human lung model used for testing. The inventors will utilize a ventilated or ventilated/perfused ex vivo lung model to test the aerosol collection system. Testing will utilize both healthy lungs obtained for transplant but found to be unusable and explanted lungs removed at the time of lung transplant. Explants from COPD, IPF, and CF patients will be included in AIM 2, if they are available, since these are likely to have distinct microbial communities.

Use of LPS induced lung injury in an ex vivo lung model to explore measurements of host response biomarkers in exhaled aerosol samples. The inventors will induce lung injury by infusing LPS into the perfusate of the ex vivo model using techniques previously described and collect exhaled aerosol before, during, and after infusion. These studies will allow the inventors to determine the extent to which biomarkers of host response, lung injury, inflammation, and permeability are detectable within exhaled aerosol samples.

### C) Approach:

Aim 1: To develop a highly efficient device for collecting exhaled aerosols from the lungs of mechanically ventilated patients.

Introduction: Aerosols are generated in the proximal lung by high-speed airflows passing over secretions in the large airways during exhalation and in the distal lung by the rapid opening of small airways during inhalation. Aerosols generated in the distal lung will be smaller than those generated in the proximal lung because the lung filters out larger aerosols as they are exhaled through the airway tree. Aerosols generated in the proximal lung, throat, and mouth are subject to less filtering and therefore may be in larger sizes when exhaled. Exhaled aerosols from the distal lung are typically ˜1-10 μm in diameter while aerosols from the proximal lung may be much larger ˜100's μm. Collecting aerosols based on their size therefore, in principle, provides a means of selectively sampling the distal lung. The selectivity of this sampling along with the availability of microbial DNA and biomarkers within these aerosol samples will be validated through the proposed research.

Design: The inventors have developed a 2-stage, impaction-based prototype aerosol collector that has undergone preliminary testing as described herein. Both impaction stages in certain designs disclosed herein (housed within PVC pipe) use a nozzle and a conical impaction surface to collect aerosol droplets and funnel them into an Eppendorf tube. For impaction, collection efficiency increases with Stokes number=ρd²V/18 μD where ρ is the density of the aerosol, d is aerosol size, V is air velocity, μ is air viscosity, and D is the offset between the nozzle and the collection surface. The first stage is designed to collect larger aerosols generated in the large proximal airways. It has a large offset between the nozzle and the impaction surface. Larger droplets with high inertia will impact and collect in the funnel providing a “proximal lung” sample while smaller droplets will remain entrained in the airflow passing into stage 2. Stage 2 has a much smaller offset between the nozzle and the impaction surface making it a highly efficient collector of even small aerosols. These aerosols are funneled into a second Eppendorf providing a “distal lung” sample. Air then exits the device back into the ventilator. Eppendorf tubes are contained within threaded holders that screw into each stage ensuring that the collector is airtight and able to maintain positive end-expiratory pressure (PEEP) when applied as part of mechanical ventilation.

Preliminary Data: The inventors tested the collection efficiency of the device using two saline test aerosols delivered into the collector with a steady 20 L/min air flow from an air compressor. The inventors generated a small test aerosol using an Aerogen Solo electronic (vibrating mesh) medical nebulizer that had a volume median diameter of 4.3±0.1 μm and a large test aerosol using a syringe and nasal irrigation cannula which had a volume median diameter of 200±188 μm, as measured by laser diffraction (Malvern Mastersizer S). The inventors also measured upstream back pressure caused by the device. The inventors demonstrated good collection efficiencies and specificity of collection by stage with large aerosols being collected primarily in the proximal lung stage and smaller aerosols being collected primarily in the distal lung stage of the device, as shown in Table 1 above.

The inventors also tested whether the collector would affect normal ventilator operation by placing it in the exhalation line of a mechanical ventilator circuit including a Puritan Bennett 980 series Ventilator and a Michigan Test lung. The collector had only minor effects on delivered tidal volume and airway pressures but did decrease exhalation flowrates. The inventors are revising the design to decrease resistance and minimize this effect to avoid the potential for breath stacking at high ventilatory rates.

Approach: In AIM 1 the inventors will tune the device to maximize collection efficiency while minimizing exhalation resistance using methods like those described in the preliminary data. All candidate designs will be tested in a ventilator circuit including a Puritan Bennett 980 ventilator connected to a Michigan test lung to see if they substantially affect delivered tidal volume, pressure, rate, and inhalation/exhalation flowrate under varying ventilator and test lung settings, simulating different specific patient conditions.

The inventors will validate the selective collection of proximal and distal lung samples using a ventilated and perfused ex vivo human lung model (see, e.g., FIGS. 14A-14C). The inventors will add a small amount (10 mCi) of Technetium 99m pertechnetate to the perfusate (MW=186 Da) and allow it to circulate through the lungs for 30 minutes. This probe is small enough to diffuse into the liquid on the surface of the alveoli. The bronchial circulation will not be perfused. Will also use a bronchoscope to spot fluorescently labeled (70 kDa) dextran dye onto different portions of the airway tree. The inventors will collect exhaled aerosol while the ex vivo lungs are ventilated over a period of 6 hours. Eppendorf tubes will be changed out once per hour in the distal and proximal collector stages. Sample radioactivity and fluorescence will be assessed in each. Fluorescence associated with the dye (as measured by spectrofluorimetry) will be indicative of material originating from the airways. Radioactivity associated with the Technetium 99m will be associated with material originating from the alveoli and the respiratory and terminal bronchioles in communication with the alveoli. The inventors will estimate the proximal and distal lung contribution to both stage samples and repeat the experiment 3 times.

Alternative approaches: One goal is to design a passive device that depends on the patients' exhaled breath velocity to drive deposition of the aerosol with a low enough resistance that it does not affect vent function. However, it is possible that higher velocities will be required for efficient collection by impaction, or that device resistance may be too high. If either of these cases occur, the inventors will consider the addition of an in-line suction fan to generate higher velocities in the impactor stages. The inventors will also consider the use of electrostatic charge imparted onto components of the collector either passively, based on the use of materials that have a charge imparted onto them during the manufacturing process, or actively though a conducting surface and battery, to increase collection efficiency.

Aim 2: To compare quantitative assessments of the bacterial and fungal communities in distal exhaled aerosol to BAL in ex vivo lungs.

Preliminary data: The inventors received IRB approval to collect exhaled aerosol samples from healthy subjects. One subject exhaled through the collector five times for a period of 45-60 minutes each. The average liquid mass collected in the proximal stage was 237±213 mg and the distal stage was 52±17 mg (±SD). During one trial, prior to collection, the inventors swabbed the interior surfaces of the collector to examine for background contamination, and then the inventors swabbed the interior again at the end of the experiment.

General Approach: The inventors will measure bacterial and fungal DNA sequencing yields of the resident microbiome in exhaled aerosols samples collected during the ventilation of ex vivo human lungs. The inventors will test 10 lung pairs or more, depending on whether outcomes require design changes to the collector. Proximal (large airways) and distal (small airway and alveolar) samples will be independently collected. Comparisons will be made against bronchoalveolar lavage (distal sample) and endotracheal aspirate (proximal sample) collected at the end of the experiment, as gold standards. The inventors will primarily utilize lungs made available for transplant but not used since these are the most available and previous studies have demonstrated the presence of bacterial communities in parenchymal tissue samples from these lungs. The inventors will also perform studies using explanted lungs from COPD, IPF, and CF patients, as they become available. The inventors will begin by collecting a 4-hour sample. The inventors will then increase or decrease the collection interval depending on signal from the samples. The inventors will utilize 16S based methods with traditional cultures also being performed for comparison purposes.

Biomarker analysis: In support of the studies to be pursued in AIM 3 the inventors will also assess candidate biomarkers of host response and lung injury including: RAGE, SPD (epithelial dysfunction), ANG-2 (endothelial dysfunction), IL-6, IL-8, IL-10, TNFR1, ST2, GDF15 (inflammation), and procalcitonin/pentraxin-3 (bacterial infection) in these samples.

16S rRNA Gene and Internal Transcribed Spacer (ITS) Region Sequencing. Since the inventors anticipate low microbial biomass samples, special care will be taken to avoid the introduction of exogenous DNA and to measure potential background contaminants. Genomic DNA will be extracted using the DNeasy PowerSoil kit (Qiagen). As a bead-beating approach, this method can be used to extract bacterial and fungal nucleic acids. The inventors will sequence the V4 region of the 16S rRNA gene to identify bacteria and the internal transcribed spacer (ITS) region of the nuclear ribosomal RNA gene to identify fungi. Genomic DNA will be sized and measured for quality by gel electrophoresis and fluorometrically quantified (Qubit fluorometric quantitation). For 16S and ITS sequencing, DNA will be PCR amplified in triplicate (to ensure rigor and reproducibility). PCR master mixes will be treated with nucleases to reduce potential background from contaminants (ArcticZymes Technologies, Norway). Inline indexed (barcoded) V4 16S primers will be used for bacterial amplification and fungal species will be interrogated using sequences derived from the ITS region in an analogous fashion using primers targeting the ITS2 region. For data rigor and reproducibility, each experimental step (DNA extraction and PCR) will include reagent blanks as negative controls, and mock community standard (ZymoBIOMICS™, Zymo Research) as positive controls. Amplicons will be cleaned, normalized, and pooled. Library concentrations will be calculated, normalized, pooled, and run on the Illumina MiSeq platform with v3 (600 cycle) kits. Sequencing will be completed to generate at least 10,000 high quality reads per sample.

16S and ITS Taxonomic Profiles. The 16S and ITS reads will be processed with standard in-house quality control (QC) pipeline that includes quality trimming, low complexity filtering, paired-end assembly, followed by additional paired-assembly QC. Resulting paired reads will be processed through a Mothur-dependent in-house pipeline that performs alignment with Ribosomal Database Project (RDP) rRNA sequences and ITS reads will be classified using the UNITE ITS database. The pipeline also generates Operational Taxonomic Units (OTUs). Results from this 16S/ITS pipeline will be converted into per sample taxonomic profiles represented as categorical counts in matrices of dimension (number of samples)×(number of categories) for subsequent use in analyses described below.

Environmental Controls: At each time point, samples will be taken to measure exogenous background contamination. For BAL samples, a control sample (saline flushed through the bronchoscope) will also be obtained. A similar process will be performed with the catheters used to sample endotracheal aspirate. Taxonomic distribution patterns from the environmental controls will be used to identify contaminants in the experimental samples by fitting a mixture model. The mixture model assumes that the sequenced observed sample is a combination of a hypothetical clean (no exogenous contamination) and the empirically determined environmental control sample, at a proportion that is unknown but can be computed with an optimization algorithm.

Exploratory use of point of care metagenomics analysis: The inventors will include the application of the Nanopore MINION® to assess the feasibility of real time measurements using techniques similar to previous studies. This device weighs <500 g, is portable, connects directly through USB, and may allow for point of care sequencing in the future. The inventors will also consider application of the M1kC device, which is essentially the next generation version of the MINION®. As metagenomic sequencing is not dependent on PCR primers, this approach will allow the inventors to examine taxonomic profiles that include bacteria, fungi, and viruses simultaneously with increased granularity (e.g., species level). This approach will also provide information on the functional potential of the microbiome such as the composition of antibiotic resistance genes.

Hypothesis testing: The inventors will assess the microbiota in exhaled aerosol samples and compare them to microbiota in endotracheal aspirate and BAL. The inventors hypothesize similarity between BAL and the distal lung aerosol samples and between the endotracheal aspirate and the proximal lung aerosol samples. The inventors further hypothesize that there will be substantial differences between the proximal and distal lung samples indicating distinct communities in the bronchial airways vs. small airways and alveoli. Comparisons will be performed using distance-based, abundance-based, and distribution-based approaches where each method accounts for the compositional nature of the sequence data and provides opportunities to integrate and determine associations with other experimental variables through statistical models. As these are matched samples (from the same lungs), paired difference analyses will be performed to examine the degree of differences between the two samples methods (BAL and aerosol) from the same subject by computing the difference (delta) in taxonomic abundance, overall composition, or diversity between the two samples from the same lung that can be incorporated into linear models.

Aim 3: To measure temporal changes in biomarkers of host response in exhaled aerosol samples collected from an ex vivo human lung model during an LPS induced injury.

Introduction and previous work: The goal of Aim 3 is to evaluate the utility of the respiratory aerosol collector for studying temporal changes in biomarkers associated with lung injury and host response. The inventors will use a ventilated and perfused ex vivo human lung model with a lipopolysaccharide (LPS) induced injury to assess changes in candidate biomarkers related to epithelial and endothelial permeability, inflammation, and bacterial infection. Biomarker measurements in Aim 2 studies will be used to determine the sampling time needed for repeatable measurements of the preliminary biomarker panel which includes: albumin (lung permeability), RAGE, SPD (epithelial dysfunction), ANG-2 (endothelial dysfunction), IL-6, IL-8, IL-10, TNFR1, ST2, GDF15 (inflammation), and procalcitonin/pentraxin-3 (bacterial infection).

Ex vivo lung perfusion has been used for some time to prepare and evaluate organs for lung transplant. The ex vivo model is shown in FIGS. 14A-14C and FIG. 15. The lungs are ventilated using a clinical ventilator and while perfusion is provided perfused by a bypass pump. The perfusate includes electrolytes to match human plasma, human albumin, and glucose and is heated to 38 deg. C. The ex vivo lung model with LPS induced lung injury has been used to test anti-inflammatory therapies. In these studies, LPS was delivered over time into the pulmonary artery.

Cytokine levels were assessed in the perfusate at t=0, 2, 4, and 6 hours, demonstrating expected inflammatory response to LPS, including increases in IL-1β, IL-6, IL-10, TNFα, IL-8, INF-γ, IL-12, IL-4, IL-13, and IL-2. BAL was also collected at the end of the experiment and used to assess leaked albumin which decreased by 20-fold with anti-inflammatory therapy. Immune response in these models is assumed to be associated with resident immune cells within the donated lungs.

General study design: In this pilot study the inventors will test 3 lung pairs with LPS induced injury. Inclusion criteria: pO2>250 mmHg, <20 pack years tobacco history, <6 hours cold ischemic time, no previously diagnosed lung disease, and not suitable for transplantation. Details on the establishment of the ex vivo model are available in. LPS (E. coli 0111:B4 endotoxin 5 μg/kg of donor estimated weight in 20 ml of PBS) will be infused into the pulmonary artery. Aerosol will be continuously collected while the lungs are mechanically ventilated. Each aerosol sample will include one hour of collection. Perfusate samples will be collected in parallel to exhaled aerosol samples. Biomarkers in the preliminary panel described above will be assessed in both aerosol samples and the perfusate. Perfusate samples will also be assessed in a blood gas analyzer. Lung weights will be continually assessed during the study using a scale underneath each lung. The inventors will also collect biopsy samples via bronchoscopy during the testing. After 6 hours after LPS delivery, bronchoalveolar lavage (BAL) will be performed. Candidate biomarkers will be assessed in the BAL using methods similar to those previous reported. The experiment will then be concluded, and further tissue samples will be collected for histology.

Expected outcomes and analysis: The inventors will consider time-based changes in proposed biomarkers in both distal and proximal aerosol samples over time, before, during, and after the initiation of lung injury. The inventors anticipate that the initiation LPS infusion will be rapidly followed by indications of increased inflammatory biomarkers in the exhaled aerosol samples. The inventors expect that these markers will be detectable in exhaled aerosol prior to being detectable in perfusate. The inventors then expect a succession of increases in indicators of epithelial and endothelial injury, followed finally by the detection of albumin in the exhaled aerosol (which permeates from the perfusate into the air spaces). The goals of this aim are to determine: (1) the sampling times required to provide consistently quantifiable measurements of the biomarkers, (2) which biomarkers provide the earliest and strongest signals associated with lung injury, (3) whether the sampling of the aerosol illustrates temporal biomarker changes anticipated with the evolution of inflammation, lung injury, loss of barrier integrity, and decreased oxygenation, (4) the relationship between aerosol, perfusate, and BAL biomarker measurements, (5) which biomarkers provide best indication of lung injury indicated vis histology, wet/dry weight. These results will facilitate the design of more comprehensive studies with ex vivo lungs and pilot studies with mechanically ventilated subjects.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An impactor sample collection apparatus, comprising: an inlet; a first impaction stage downstream of the inlet, the first impaction stage defining a longitudinal first axis; a second impaction stage downstream of the first impaction stage, the second impaction stage being offset from the first impaction stage along a second axis that is perpendicular to the longitudinal first axis; and an outlet downstream of the second impaction stage.

Example 2. The impactor sample collection apparatus of any example herein, particularly claim 1, wherein the first impaction stage comprises a nozzle configured to accelerate a flow of gas through the first impaction stage along the longitudinal first axis.

Example 3. The impactor sample collection apparatus of any example herein, particularly example 1 or example 2, wherein the first impaction stage is configured to receive a removable sample collection container.

Example 4. The impactor sample collection apparatus of any example herein, particularly example 3, wherein the first impaction stage further comprises a funnel between the nozzle and the sample collection container.

Example 5. The impactor sample collection apparatus of any example herein, particularly any one of examples 1-4, wherein the impactor sample collection apparatus comprises a conduit having a first conduit portion that includes the first impaction stage, and a second conduit portion that extends along the second axis and interconnects the first impaction stage and the second impaction stage.

Example 6. The impactor sample collection apparatus of any example herein, particularly example 5, wherein when a sample collection container is received in the first impaction stage, the second conduit portion is above the sample collection container.

Example 7. The impactor sample collection apparatus of any example herein, particularly any one of examples 1-6, wherein the first impaction stage and the second impaction stage are parallel or substantially parallel.

Example 8. The impactor sample collection apparatus of any example herein, particularly any one of examples 1-7, wherein the second impaction stage further comprises a nozzle, and a funnel downstream of the nozzle.

Example 9. The impactor sample collection apparatus of any example herein, particularly example 8, wherein the second impaction stage further comprises an impaction member positioned in a flow path of the second impaction stage.

Example 10. The impactor sample collection apparatus of any example herein, particularly example 9, wherein the impaction member is downstream of the nozzle of the second impaction stage.

Example 11. The impactor sample collection apparatus of any example herein, particularly any one of examples 8-10, wherein the nozzle of the second impaction stage is received in the funnel of the second impaction stage.

Example 12. A mechanical ventilator comprising the impactor sample collection apparatus of any example herein, particularly any one of examples 1-11 coupled to an exhalation limb of the mechanical ventilator.

Example 13. A method, comprising collecting aerosolized liquid droplets from exhaled breaths of a patient in the first impaction stage and in the second impaction stage with the impactor sample collection apparatus of any example herein, particularly any one of examples 1-11.

Example 14. An impactor sample collection apparatus, comprising: an inlet; a first impaction stage downstream of the inlet, the first impaction stage configured to receive a removable sample collection container; a second impaction stage downstream of the first impaction stage, the second impaction stage configured to receive a removable sample collection container; and an outlet downstream of the second impaction stage.

Example 15. The impactor sample collection apparatus of any example herein, particularly example 14, wherein the first impaction stage defines a longitudinal first axis, and the second impaction stage is offset from the first impaction stage along a second axis that is perpendicular to the longitudinal first axis.

Example 16. The impactor sample collection apparatus of any example herein, particularly any one of example 14 or example 15, wherein the first impaction stage comprises a nozzle configured to accelerate a flow of gas through the first impaction stage.

Example 17. The impactor sample collection apparatus of any example herein, particularly example 16, wherein the first impaction stage further comprises a funnel between the nozzle and the sample collection container.

Example 18. The impactor sample collection apparatus of any example herein, particularly any one of examples 15-17, wherein the impactor sample collection apparatus comprises a conduit having a first conduit portion that includes the first impaction stage, and a second conduit portion that extends along the second axis and interconnects the first impaction stage and the second impaction stage.

Example 19. The impactor sample collection apparatus of any example herein, particularly any one of examples 14-18, wherein the second impaction stage further comprises a nozzle, and a funnel downstream of the nozzle.

Example 20. The impactor sample collection apparatus of any example herein, particularly example 19, wherein the second impaction stage further comprises an impaction member positioned in a flow path of the second impaction stage downstream of the nozzle of the second impaction stage.

Example 21. The impactor sample collection apparatus of any example herein, particularly example 20, wherein the impaction member comprises an electrically conductive material and is configured to be electrically charged.

Example 22. The impactor sample collection apparatus of any example herein, particularly any one of examples 19-21, wherein the nozzle of the second impaction stage is received in the funnel of the second impaction stage.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims and equivalents of the recited features. We therefore claim all that comes within the scope and spirit of these claims.

1. An impactor sample collection apparatus, comprising:
an inlet;
a first impaction stage downstream of the inlet, the first impaction stage defining a longitudinal first axis;
a second impaction stage downstream of the first impaction stage, the second impaction stage being offset from the first impaction stage along a second axis that is perpendicular to the longitudinal first axis; and
an outlet downstream of the second impaction stage;
wherein the first impaction stage is configured to receive a removable sample collection container.

2. The impactor sample collection apparatus of claim 1, wherein the first impaction stage comprises a nozzle configured to accelerate a flow of gas through the first impaction stage along the longitudinal first axis.

3. The impactor sample collection apparatus of claim 2, wherein the second impaction stage is configured to receive a second removable sample collection container.

4. The impactor sample collection apparatus of claim 3, wherein the first impaction stage further comprises a funnel between the nozzle and the sample collection container.

5. The impactor sample collection apparatus of claim 1, wherein the impactor sample collection apparatus comprises a conduit having a first conduit portion that includes the first impaction stage, and a second conduit portion that extends along the second axis and interconnects the first impaction stage and the second impaction stage.

6. The impactor sample collection apparatus of claim 5, wherein when the sample collection container is received in the first impaction stage, the second conduit portion is above the sample collection container.

7. The impactor sample collection apparatus of claim 1, wherein the first impaction stage and the second impaction stage are parallel or substantially parallel.

8. The impactor sample collection apparatus of claim 1, wherein the second impaction stage further comprises a nozzle, and a funnel downstream of the nozzle.

9. The impactor sample collection apparatus of claim 8, wherein the second impaction stage further comprises an impaction member positioned in a flow path of the second impaction stage.

10. The impactor sample collection apparatus of claim 9, wherein the impaction member is downstream of the nozzle of the second impaction stage.

11. The impactor sample collection apparatus of claim 8, wherein the nozzle of the second impaction stage is received in the funnel of the second impaction stage.

12. A mechanical ventilator comprising the impactor sample collection apparatus of claim 1 coupled to an exhalation limb of the mechanical ventilator.

13. A method, comprising collecting aerosolized liquid droplets from exhaled breaths of a patient in the first impaction stage and in the second impaction stage with the impactor sample collection apparatus of claim 1.

14. An impactor sample collection apparatus, comprising:
an inlet;
a first impaction stage downstream of the inlet, the first impaction stage configured to receive a removable sample collection container;
a second impaction stage downstream of the first impaction stage, the second impaction stage configured to receive a removable sample collection container; and
an outlet downstream of the second impaction stage.

15. The impactor sample collection apparatus of claim 14, wherein the first impaction stage defines a longitudinal first axis, and the second impaction stage is offset from the first impaction stage along a second axis that is perpendicular to the longitudinal first axis.

16. The impactor sample collection apparatus of claim 14, wherein the first impaction stage comprises a nozzle configured to accelerate a flow of gas through the first impaction stage.

17. The impactor sample collection apparatus of claim 16, wherein the first impaction stage further comprises a funnel between the nozzle and the sample collection container.

18. The impactor sample collection apparatus of claim 15, wherein the impactor sample collection apparatus comprises a conduit having a first conduit portion that includes the first impaction stage, and a second conduit portion that extends along the second axis and interconnects the first impaction stage and the second impaction stage.

19. The impactor sample collection apparatus of claim 14, wherein the second impaction stage further comprises a nozzle, and a funnel downstream of the nozzle.

20. The impactor sample collection apparatus of claim 19, wherein the second impaction stage further comprises an impaction member positioned in a flow path of the second impaction stage downstream of the nozzle of the second impaction stage.

21. The impactor sample collection apparatus of claim 20, wherein the impaction member comprises an electrically conductive material and is configured to be electrically charged.

22. The impactor sample collection apparatus of claim 19, wherein the nozzle of the second impaction stage is received in the funnel of the second impaction stage.
